SEQUENCE LISTING (26 sequences)

SEQ ID NO: 1 - DNA, 2535 bp, HIV-1 133
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaacaacaacacaagaagaagtatacatataggaccagggagcgcattttatacaacaggacaaataataggagatataagacaagcacattgtaacattaatggaacacaatggaataaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtatataaaaatattcataatgatagtaggaggcttgataggtttaagaatagtttttactgtactttctatagtgaatagagttaggcagggatactcaccattatcgttggagacccgcctcccaacccagaggggacccgacaggcccgaaggaatcgaagaagaaggtggagagagagacagagacacatccggtcaattagtgaatggattcttagcaattatctgggtcgacctgcggagcctgtgcctcttcagctaccaccgcttgagagacttactcttgattgtaacgaggattgtggaacttctgggacgcagggggtgggaagccctcaagtatttgtggaatctcctccagtattggagtcaggaactaaagaagagtgctgttagcttgttcaatgccatagccatagcagtagctgaggggacagataggataatagaagtaatacaaagggcttatagggccattctccacatacctagaagaataagacagggcttggaaaggcttttgctataa

SEQ ID NO: 2 - DNA, 2535 bp, HIV-1 mutant g12
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtatataaaaatattcataatgatagtaggaggcttgataggtttaagaatagtttttactgtactttctatagtgaatagagttaggcagggatactcaccattatcgttggagacccgcctcccaacccagaggggacccgacaggcccgaaggaatcgaagaagaaggtggagagagagacagagacacatccggtcaattagtgaatggattcttagcaattatctgggtcgacctgcggagcctgtgcctcttcagctaccaccgcttgagagacttactcttgattgtaacgaggattgtggaacttctgggacgcagggggtgggaagccctcaagtatttgtggaatctcctccagtattggagtcaggaactaaagaagagtgctgttagcttgttcaatgccatagccatagcagtagctgaggggacagataggataatagaagtaatacaaagggcttatagggccattctccacatacctagaagaataagacagggcttggaaaggcttttgctataa

SEQ ID NO: 3 - DNA, 1494 bp, HIV-1 mutant g12
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaaga

SEQ ID NO: 4 - DNA, 2007 bp, HIV-1 mutant g12
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtat

SEQ ID NO: 5 - DNA, 2535 bp, HIV-1 mutant g112
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaatcagaaaacaataatctttaggcagtcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtatataaaaatattcataatgatagtaggaggcttgataggtttaagaatagtttttactgtactttctatagtgaatagagttaggcagggatactcaccattatcgttggagacccgcctcccaacccagaggggacccgacaggcccgaaggaatcgaagaagaaggtggagagagagacagagacacatccggtcaattagtgaatggattcttagcaattatctgggtcgacctgcggagcctgtgcctcttcagctaccaccgcttgagagacttactcttgattgtaacgaggattgtggaacttctgggacgcagggggtgggaagccctcaagtatttgtggaatctcctccagtattggagtcaggaactaaagaagagtgctgttagcttgttcaatgccatagccatagcagtagctgaggggacagataggataatagaagtaatacaaagggcttatagggccattctccacatacctagaagaataagacagggcttggaaaggcttttgctataa

SEQ ID NO: 6 - DNA, 1494 bp, HIV-1 mutant g112
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaatcagaaaacaataatctttaggcagtcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaaga

SEQ ID NO: 7 - DNA, 2007 bp, HIV-1 mutant g112
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaatcagaaaacaataatctttaggcagtcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtat

SEQ ID NO: 8 - DNA, 2535 bp, HIV-1 mutant g14
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgaccagttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattaatggaacacaatggaataaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtatataaaaatattcataatgatagtaggaggcttgataggtttaagaatagtttttactgtactttctatagtgaatagagttaggcagggatactcaccattatcgttggagacccgcctcccaacccagaggggacccgacaggcccgaaggaatcgaagaagaaggtggagagagagacagagacacatccggtcaattagtgaatggattcttagcaattatctgggtcgacctgcggagcctgtgcctcttcagctaccaccgcttgagagacttactcttgattgtaacgaggattgtggaacttctgggacgcagggggtgggaagccctcaagtatttgtggaatctcctccagtattggagtcaggaactaaagaagagtgctgttagcttgttcaatgccatagccatagcagtagctgaggggacagataggataatagaagtaatacaaagggcttatagggccattctccacatacctagaagaataagacagggcttggaaaggcttttgctataa

SEQ ID NO: 9 - DNA, 1494 bp, HIV-1 mutant g14
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgaccagttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattaatggaacacaatggaataaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaaga

SEQ ID NO: 10 - DNA, 2007 bp, HIV-1 mutant g14
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgaccagttctcgaacaatgctaaaaccataatagtacaactgacagaacctgtagtaatcaattgtataagacccaaccagaacacaagaagaagtatacatataggaccagggagcgcattttatacaacaggacaaataataggagatataagacaagcacattgtaacattaatggaacacaatggaataaaactttaagtctaatagttgataaattaaagaaacaatttaataataaaacaataatctttaggaactcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtat

SEQ ID NO: 11 - protein, 844 aa, HIV-1 133
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNNNTRRSIHIGPGSAFYTTGQIIGDIRQAHCNINGTQWNKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWYIKIFIMIVGGLIGLRIVFTVLSIVNRVRQGYSPLSLETRLPTQRGPDRPEGIEEEGGERDRDTSGQLVNGFLAIIWVDLRSLCLFSYHRLRDLLLIVTRIVELLGRRGWEALKYLWNLLQYWSQELKKSAVSLFNAIAIAVAEGTDRIIEVIQRAYRAILHIPRRIRQGLERLLL

SEQ ID NO: 12 - protein, 844 aa, HIV-1 mutant g12
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNQNTRRSIHIGPGSAFYTTGQIIGDIRQAHCNIQGTQWQKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWYIKIFIMIVGGLIGLRIVFTVLSIVNRVRQGYSPLSLETRLPTQRGPDRPEGIEEEGGERDRDTSGQLVNGFLAIIWVDLRSLCLFSYHRLRDLLLIVTRIVELLGRRGWEALKYLWNLLQYWSQELKKSAVSLFNAIAIAVAEGTDRIIEVIQRAYRAILHIPRRIRQGLERLLL

SEQ ID NO: 13 - protein, 498 aa, HIV-1 mutant g12
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNQNTRRSIHIGPGSAFYTTGQIIGDIRQAHCNIQGTQWQKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKR

SEQ ID NO: 14 - protein, 669 aa, HIV-1 mutant g12
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNQNTRRSIHIGPGSAFYTTGQIIGDIRQAHCNIQGTQWQKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWY

SEQ ID NO: 15 - protein, 844 aa, HIV-1 mutant g112
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNQNTRRSIHIGPGSAFYTTGQIIGDIRQAHCNIQGTQWQKTLSLIVDKLKKQFNQKTIIFRQSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWYIKIFIMIVGGLIGLRIVFTVLSIVNRVRQGYSPLSLETRLPTQRGPDRPEGIEEEGGERDRDTSGQLVNGFLAIIWVDLRSLCLFSYHRLRDLLLIVTRIVELLGRRGWEALKYLWNLLQYWSQELKKSAVSLFNAIAIAVAEGTDRIIEVIQRAYRAILHIPRRIRQGLERLLL

SEQ ID NO: 16 - protein, 498 aa, HIV-1 mutant g112
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNQNTRRSIHIGPGSAFYTTGQIIGDIRQAHCNIQGTQWQKTLSLIVDKLKKQFNQKTIIFRQSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKR

SEQ ID NO: 17 - protein, 669 aa, HIV-1 mutant g112
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNQNTRRSIHIGPGSAFYTTGQIIGDIRQAHCNIQGTQWQKTLSLIVDKLKKQFNQKTIIFRQSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWY

SEQ ID NO: 18 - protein, 844 aa, HIV-1 mutant g14
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDQFSNNAKTIIVQLTEPVVINCIRPNQNTRRSIHIGPGSAFYTTGQIIGDIRQAHCNINGTQWNKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWYIKIFIMIVGGLIGLRIVFTVLSIVNRVRQGYSPLSLETRLPTQRGPDRPEGIEEEGGERDRDTSGQLVNGFLAIIWVDLRSLCLFSYHRLRDLLLIVTRIVELLGRRGWEALKYLWNLLQYWSQELKKSAVSLFNAIAIAVAEGTDRIIEVIQRAYRAILHIPRRIRQGLERLLL

SEQ ID NO: 19 - protein, 498 aa, HIV-1 mutant g14
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDQFSNNAKTIIVQLTEPVVINCIRPNQNTRRSIHIGPGSAFYTTGQIIGDIRQAHCNINGTQWNKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKR

SEQ ID NO: 20 - protein, 669 aa, HIV-1 mutant g14
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDQFSNNAKTIIVQLTEPVVINCIRPNQNTRRSIHIGPGSAFYTTGQIIGDIRQAHCNINGTQWNKTLSLIVDKLKKQFNNKTIIFRNSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWY

SEQ ID NO: 21 - DNA, 2535 bp, HIV-1 mutant g22
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaacaacaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaatcagaaaacaataatctttaggcagtcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtatataaaaatattcataatgatagtaggaggcttgataggtttaagaatagtttttactgtactttctatagtgaatagagttaggcagggatactcaccattatcgttggagacccgcctcccaacccagaggggacccgacaggcccgaaggaatcgaagaagaaggtggagagagagacagagacacatccggtcaattagtgaatggattcttagcaattatctgggtcgacctgcggagcctgtgcctcttcagctaccaccgcttgagagacttactcttgattgtaacgaggattgtggaacttctgggacgcagggggtgggaagccctcaagtatttgtggaatctcctccagtattggagtcaggaactaaagaagagtgctgttagcttgttcaatgccatagccatagcagtagctgaggggacagataggataatagaagtaatacaaagggcttatagggccattctccacatacctagaagaataagacagggcttggaaaggcttttgctataa

SEQ ID NO: 22 - DNA, 2007 bp, HIV-1 mutant g22
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaacaacaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaatcagaaaacaataatctttaggcagtcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaagagcagtgggaatgctaggagccatgttccttgggttcttgggagcagcaggaagcactatgggcgcagcgtcactggcgttgacggtacaggccagacaagtattgtctggtatagtgcaacagcagaacaatttgctgagggctattgaggcgcaacagcacctgttgcaactcacagtctggggcatcaagcagctccaggcaagagtcctggctgtggaaaggtacctaggggatcaacagctcctgggaatttggggttgctctggaaaactcatctgcaccactgctgtgccttggaatgatagttggagtaataaatctctgaaatatatttgggataacatgacctggatgcagtgggaaaaagaaattgacacccacataggcacaatataccaattaattgaagaatcgcagtaccaacaagaaaagaatgaaaaagaattattggaactagataaatgggcaagtttgtggaattggtttgacataacaaactggctgtggtat

SEQ ID NO: 23 - DNA, 1494 bp, HIV-1 mutant g22
atgagagtgaaggagatcaggaggaattatcagcacttgtggagatggggcacgatgctccttgggatgttcatgatctgtagtgcagaagaacaattgtgggtcacagtctattatggggtacctgtgtggaaagaagcaactaccactctattttgtgcatcagatgctaaagcatatgatacagaggtacataatgtctgggccacacatgcctgtgtacccacagaccccaacccacgagaagtagtaatgggaaatgtgacagaagaatttaacatatggaacaatagcatggtagaacagatgcatgaagatataattagtttatgggatcaaagtctaaagccatgtgtaaaattaaccccactctgtgttactttaaattgcactaactacaatagtactagtaatgatactaccaaggaaacaggaataaaaaaatgctctttcaatatcaccacctcaggcgtaaaagataggtttacgaaggaaaatgcacttttgtatacagctgatgtagtaccaatagataatagtagtatcaactataggttgataggttgtaacacctcagtcattacccaggcctgtccaaaggtatcctttgagccaattcccatacattattgtgccccggctggttttgcgattctaaagtgtaataatgaaacattcgatggaaaaggaccatgtacaaatgtcagcacagtacaatgtacacatggaattagaccagtagtatcaactcaactgctgttaaatggcagtctagcagaaacagaggtagtaattagatctgacaatttctcgaacaatgctaaaaccataatagtacaactgacagaacctgtagtaatcaattgtataagacccaacaacaacacaagaagaagtatacatataggaccagggagcgcattttatacaacaggacaaataataggagatataagacaagcacattgtaacattcagggaacacaatggcagaaaactttaagtctaatagttgataaattaaagaaacaatttaatcagaaaacaataatctttaggcagtcctcaggaggggacccagaaattgtaatgcacagttttaactgtggaggggaatttttctactgtaatacaacacaactgtttaatagtatttggatgcttaataatacttgggatggtactaaagaggaaaatattacactcccatgcagaataaaacaaattataaacatgtggcaggaagtaggaaaagcaatgtatgcccctcccatcaaaggacaaattagatgttcatcaaacattacagggctgctattaacaagagatggtggtggtaacacgagcagcgataatgagaccttcagacctggaggaggagatatgagggataattggagaagtgaattatataaatataaagtagtaaaaattgaaccattaggagtagcacccaccaaggcaaggagaagagtggtgcagagagaaaaaaga

SEQ ID NO: 24 - protein, 844 aa, HIV-1 mutant g22
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNNNTRRSIHIGPGSAFYTTGQIIGDIRQAHCNIQGTQWQKTLSLIVDKLKKQFNQKTIIFRQSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWYIKIFIMIVGGLIGLRIVFTVLSIVNRVRQGYSPLSLETRLPTQRGPDRPEGIEEEGGERDRDTSGQLVNGFLAIIWVDLRSLCLFSYHRLRDLLLIVTRIVELLGRRGWEALKYLWNLLQYWSQELKKSAVSLFNAIAIAVAEGTDRIIEVIQRAYRAILHIPRRIRQGLERLLL

SEQ ID NO: 25 - protein, 669 aa, HIV-1 mutant g22
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNNNTRRSIHIGPGSAFYTTGQIIGDIRQAHCNIQGTQWQKTLSLIVDKLKKQFNQKTIIFRQSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKRAVGMLGAMFLGFLGAAGSTMGAASLALTVQARQVLSGIVQQQNNLLRAIEAQQHLLQLTVWGIKQLQARVLAVERYLGDQQLLGIWGCSGKLICTTAVPWNDSWSNKSLKYIWDNMTWMQWEKEIDTHIGTIYQLIEESQYQQEKNEKELLELDKWASLWNWFDITNWLWY

SEQ ID NO: 26 - protein, 498 aa, HIV-1 mutant g22
MRVKEIRRNYQHLWRWGTMLLGMFMICSAEEQLWVTVYYGVPVWKEATTTLFCASDAKAYDTEVHNVWATHACVPTDPNPREVVMGNVTEEFNIWNNSMVEQMHEDIISLWDQSLKPCVKLTPLCVTLNCTNYNSTSNDTTKETGIKKCSFNITTSGVKDRFTKENALLYTADVVPIDNSSINYRLIGCNTSVITQACPKVSFEPIPIHYCAPAGFAILKCNNETFDGKGPCTNVSTVQCTHGIRPVVSTQLLLNGSLAETEVVIRSDNFSNNAKTIIVQLTEPVVINCIRPNNNTRRSIHIGPGSAFYTTGQIIGDIRQAHCNIQGTQWQKTLSLIVDKLKKQFNQKTIIFRQSSGGDPEIVMHSFNCGGEFFYCNTTQLFNSIWMLNNTWDGTKEENITLPCRIKQIINMWQEVGKAMYAPPIKGQIRCSSNITGLLLTRDGGGNTSSDNETFRPGGGDMRDNWRSELYKYKVVKIEPLGVAPTKARRRVVQREKR

(19) United States
(12) Patent Application Publication
(10) Pub. No.: US 2005/0065320 A1
(43) Pub. Date: Mar. 24, 2005

(54) Mutated env gene, mutated env glycoprotein and the use thereof

(76) Inventors: Frederic Bedin, Lyon (FR); Frederic Beynard, Lyon (FR); Bernard Verbier, Mornant (FR); Yasemin Ataman-Onal, Lyon (FR)

Correspondence Address: Oliff & Berridge, PO Box 19928, Alexandria, VA, 22320, US

(21) Appl. No.: 10/487,094

(22) PCT Filed: Sep. 6, 2002
(86) PCT No.: PCT/FR02/03039

(30) Foreign Application Priority Data
Sep. 6, 2001 (FR) 01/11699

(51) Int. Cl. C07K 14/16; C12Q 1/70; C07H 21/04; C12N 7/00; C12N 15/867

(52) U.S. Cl. 530/350; 435/5; 435/69.3; 435/456; 435/235.1; 435/325; 536/23.72

(57) ABSTRACT

The invention relates to a mutated env gene which codes for a mutated envelope glycoprotein of virus HIV-1. In relation to the env gene of a reference or primary infection primary isolate, said gene presents at least two mutations at the glycosylation sites which are preserved from one primary isolate to another. Each mutation consists in the replacement of an AAC or AAT codon which codes for an asparagine with a CAG or CAA codon which codes for a glutamine, the two mutations at least being selected from the following: (a) at least two mutations in the part of the env gene that codes for region C3 of the env protein; (b) at least one mutation in the part that codes for region C3 and at least one mutation in the part that codes for region V3; (c) at least one mutation in the part that codes for region C2 and at least one mutation in the part that codes for region V3 chosen from the glycosylation sites at codons 862-864 and 970-972; (d) at least one mutation in the part that codes for region C2 selected from the glycosylation sites at codons 667-669, 679-681, 700-702,763-765, 844-846 and at least one mutation in the part that codes for region V3. Alternatively, the mutated env gene consists of any one of the following sequences: SEQ ID NO: 8, SEQ ID NO: 9 or SEQ ID NO: 10, or the complementary sequences thereof. The invention also relates to the mutated Env glycoprotein and the use of the gene and the glycoprotein in a pharmaceutical composition in order to produce antibodies and to evaluate a therapeutic agent.

Acquired immunodeficiency syndrome (AIDS) is a viral condition which is of major importance in America, in Europe, in Africa and in Asia. Infected individuals exhibit severe immunodepression and the disease can still be fatal, although progress has been made due to tritherapy. The disease is most commonly transmitted by sexual contact and through intravenous drug use. The disease is transmissible from mother to child. One of the causal agents of this disease is the HIV-1 retrovirus. The HIV-1 genome has been completely characterized by Wain-Hobson et al., 1985 (22); Ratner et al., 1985 (23); Muesing et al., 1985 (24); Sanchez-Pescador et al., 1985 (25). The three most important portions of the HIV-1 genome are the gag, pol and env genes. The sequence of the env gene encodes the Env protein which is synthesized in the form of a glycosylated precursor, gp160, which is cleaved by a cellular endoprotease into two subunits, the gp120 surface protein and the gp41 transmembrane protein. These two subunits remain noncovalently linked and the native glycoprotein anchored to the cytoplasmic membrane or to the viral envelope is very probably a trimer or possibly a tetramer of the gp120/gp41 complex, stabilized by interactions at the level of the extracellular domains of the gp41 subunits (McInerney T. L et al., 1998 (5)). By virtue of its structural complexity, the Env protein is very difficult to purify in its native conformation.

The development of a vaccine against HIV-1 has focused considerable international efforts since the discovery of the virus in 1983. Today, after almost 20 years of active research, not only does a credible vaccine not exist, but many teams are still seeking to identify reasonably usable vaccine approaches. The conventional strategies, namely the use of attenuated strains or of inactivated viruses, have been excluded, the risks associated with the use of a replicative retrovirus being too great to envision use in humans. Since no documented case of a human being naturally cured of the infection exists, it has not been possible to identify the immune factors of protection with any confidence. For a certain number of viral infections, such as, for example, the measles virus or the influenza virus, the main factor of protection is the induction of a neutralizing humoral response, i.e. of antibodies whose binding to the viral particle blocks entry into the target cell. For this reason, the efforts to develop a vaccine against HIV-1 were initially concentrated on this approach aimed at inducing neutralizing antibodies. In such vaccine strategies, the viral envelope glycoprotein (Env) constitutes an immunogen of choice because it is the main, or even the only, target of the neutralizing response. However, the immunization trials carried out with the envelope proteins have to date produced very disappointing results. With such immunogens, it is in fact possible to induce high neutralizing titers with respect to laboratory strains in chimpanzee and macaque animal models, and also in healthy volunteers. However, these neutralizing antibodies which are effective against the strains adapted to culturing on transformed T lymphocyte lines (laboratory strains or TCLAs) do not protect against infection with the primary viruses which are involved in the transmission of HIV-1 (Moore J. P. and D. D. Ho, 1995 (1)).

The distinction between the laboratory-adapted strains and the primary isolates obtained by short-term culturing on primary T cells emerged in 1995 and became clearer with the discovery of the coreceptors for HIV-1, of which the main two are the CCR5 and CXCR4 chemokine receptors. The vast majority of the primary viruses use the CCR5 coreceptor when they enter the target cell, in addition to the CD4 receptor common to all the viruses. The adapting to lymphoid T lines results in a loss of the ability to use CCR5 and the acquisition of the ability to use CXCR4, if the virus does not already possess it (Trkola A. et al, 1996 (2)). The other fundamental difference between the two types of virus is of the immunological type: the primary isolates are, in general, much more resistant than the laboratory strains to neutralization by antibodies and soluble forms of CD4. Specifically, epitopes such as the CD4 receptor-binding site and the V3 loop are relatively inaccessible on the Env proteins of primary isolates, whereas they are exposed at the surface of the culture-adapted viruses. Consequently, the antibodies specific for these antigenic determinants effectively neutralize the adapted strains but not the primary isolates (1). In addition, the resistance of the primary isolates to the neutralization is independent of the differential use of coreceptors between the adapted and primary strains. The rare primary isolates which use only CXCR4 as coreceptor are as difficult to neutralize as those which use CCR5. Moreover, in animal models, it is effectively possible to obtain protection against the laboratory-adapted strains, but this protection remains limited to the autologous strain and to a small number of variants which are close to it. The neutralizing response remains ineffective against heterologous strains, even if they are culture-adapted variants. This limited protection is probably due to the induction of antibodies directed against the V3 loop, which is a hypervariable region of the envelope protein. Using mixtures of immunogens derived from different isolates, it has been possible to broaden the specificity of the immune response; however, it has never been possible to induce a broad-spectrum protection covering several subtypes (Girard, M. et al., 2000 (3)).

Faced with this situation, several teams have focused on the induction of an HIV-1-specific cellular immunity. In fact, it is possible to obtain, in vaccinated humans, as in animal models, cellular responses based on cytotoxic CD8⁺ T lymphocytes (CTLs) capable of killing the infected cells or of blocking therein the replication of HIV-1 by secretion of suppressor factors. However, induction of a CTL response is not achieved in more than 30 to 40% of the immunized individuals, and the responses obtained often remain directed against too restrictive a number of epitopes.

A third approach, as yet relatively unexplored, is the induction of mucosal immunity. A relatively small number of results indicate that transudation of neutralizing IgG immunoglobulins and secretion of IgA might play a role in exclusion of the virus through the epithelia.

The particularities of HIV-1, namely replication in the cells of the immune system and the initial failure of the antiviral immune response in the infected individuals, underline the need to develop novel approaches which should be aimed at inducing antibodies which bind to the neutralization epitopes conserved between the various subtypes and present on the envelope glycoproteins of the primary viruses.

For this, the inventors have set themselves several requirements for obtaining an effective preventive vaccine. It is absolutely necessary to use envelope glycoproteins derived from primary isolates and not from TCLA laboratory strains. It is necessary to be sure to present this protein to the immune system in its native oligomeric conformation. It is necessary to identify novel broad-spectrum neutralization epitopes due to the extraordinary adaptive ability of the primary viruses to mutate in order to resist neutralization by an antibody. In fact, in the humanized SCID mouse model, a therapeutic attempt consisting of the administration of a mixture of the three antibodies IgG1b12, 2G12 and 2F5 selected, in the space of one week, a viral subpopulation resistant to neutralization with each of the three antibodies (Poignard P. et al., 1999 (5)).

The HIV-1 envelope protein is highly glycosylated (50% of the total mass). The sugars present at its surface provide, inter alia, protection against the neutralizing response (Reitter J. N. et al., 1998 (6)). Many studies have shown the role that these residues play in the infectivity or the protection. However, these studies, which are far from systematic, have always taken into account the glycosylation sites either independently of one another or on particular structural elements such as the variable loops V1, V2 and V3 or the N-terminal and C-terminal ends of the protein (6-8).

In 1998, a structural model of gp120 was proposed by Kwong after crystallization of a molecule deleted of most of its variable loops and complexed with CD4s and the Fab (antigen-binding fragment) of the 17b antibody (Kwong P. D et al., 1998 (9)). Curiously, few studies have been carried out on the arrangement of the potential glycosylation sites at the surface of the oligomeric molecule. Mention may be made of the studies by X. Zhu et al., 2000 (10), which proposes a model for the completely glycosylated form of gp120, and those by R. Whyatt et al., 1998 (11) concomitant with the crystallization studies.

Thus, according to document WO-A-99/24464, the authors identified, from the crystallized structure of the HIV-1 Env protein in which the V1/V2 loops had been deleted, the various glycosylation sites capable of interacting at the same neutralization epitope, and obtained corresponding mutants. These mutants were, however, obtained from laboratory strains, with respect to which it was-previously mentioned that they are no longer suitable for the search for an effective vaccine.

In addition, with the exception of Kwong (Kwong P. D et al., 2000 (12)), all the results have essentially been obtained on TCLA strains. Now, the culture-adapted viruses have very marked epitope differences compared to the primary viruses, and the studies by Zhu and Whyatt do not reflect the exact reality of the antigenic structure of native HIV-1 gp120.

The inventors have first of all put forward and verified the hypothesis that, at the level of the trimer of heterodimers present at the surface of the infected cells or the virus, the most conserved potential glycosylation sites are grouped together so as to protect conformational elements which are important for the virus.

To test this hypothesis, they have performed a considerable study. They have positioned all the potential glycosylation sites present on the envelope sequences of 13 HIV-1 primary infection primary isolates (133, 146, 153, 159, 160, 374, 384, Qz, 120, 309, 355, 373, 426) and, by way of comparison, on 5 TCLA strains (MN, HXB2, OYI, RF, SF2). The twelve primary infection primary sequences (with the exception of QZ) were isolated and characterized in the laboratory from PBMCs. (peripheral blood mononuclear cells) from patients in the primary infection phase, hospitalized in the Croix-rousse Hospital in Lyon (Ataman-Önal Y. et al., 1999 (13)). The choice of primary infections is justified by the fact that the virus present at this stage of the disease is close to the virus which initiated the infection. The potential glycosylation sites which were conserved in the 13 reference sequences were then located and the corresponding sequences selected. These sites correspond to the protein motif N.X.T/S in which N signifies asparagine, X represents any amino acid with the exception of proline, T signifies threonine and S signifies serine. The results of this study are given in table 1.

TABLE 1
alignment of the potential glycosylation
sites on 13 primary isolate (PI) sequences from patients
in primary infection and 5 TCLA strain sequences (from
which the V1/V2 loops have been deleted)

| Regions | Aa | 133 | 146 | 153 | 159 | 160 | 374 | 384 | Qz | 120 | 309 | 355 | 373 | 426 | MN | HXB2 | OYI | RF | SF2 |
|---|---|---|---|---|---|---|---|---|---|---|---|---|---|---|---|---|---|---|---|
| C1 | 155 | + | | | | | | | | | | | | | | | | | |
| C2 | 190 | + | + | + | + | + | + | + | + | + | + | + | + | + | + | + | | + | + |
| I | 223 | + | | | + | + | | + | | | | + | | | | + | | | + |
| I | 227 | | + | + | + | | + | | + | + | | | + | + | | + | + | + | |
| I | 234 | + | + | + | + | + | + | + | + | + | + | + | + | + | + | + | + | + | + |
| I | 255 | + | + | + | + | + | + | + | + | + | + | + | + | + | + | + | + | + | + |
| I | 269 | + | + | + | + | + | + | + | + | + | + | + | + | + | + | + | + | + | + |
| v | 282 | | + | | | + | + | + | + | + | + | + | + | + | + | + | + | + | + |
| V3 | 288 | | + | + | + | + | | + | + | + | + | + | + | + | + | + | + | + | + |
| I | 294 | + | + | + | + | + | + | + | + | + | + | + | + | + | | + | + | + | + |
| v | 324 | | + | | + | + | + | + | + | + | | + | + | + | + | + | + | + | + |
| C3 | 326- | + | + | + | + | + | + | + | + | + | | | + | | + | + | | + | + |
| | 331 | | | | | | | | | | | | | | | | | | |
| I | 347 or | + | + | + | + | + | + | + | + | + | + | + | + | + | + | + | + | + | + |
| | 348 | | | | | | | | | | | | | | | | | | |
| v | 353 or | + | | + | + | + | + | + | + | | + | | + | | + | | | | + |
| | 354 | | | | | | | | | | | | | | | | | | |
| V4 | 377 | + | | | + | + | | | + | + | | + | + | + | | + | + | + | + |
| I | 383 | | + | + | + | + | + | + | + | + | + | | | + | + | + | + | + | + |
| I | 387 | | | | | | | | | | | + | | | | | + | | |
| I | 389 | + | + | + | | + | + | + | | + | | + | + | + | + | + | | + | + |
| I | 393 | | | + | + | + | + | + | + | + | + | + | | | + | | + | + | |
| I | 399 | + | | + | + | + | + | + | + | + | + | | + | | + | + | | + | + |
| v | 405 | | | + | + | | | | | | | | | | | | | | |
| C4 | 428 | | | | | | | | | | | + | | | | | | | |
| I | 432 | | | | | | | | + | | | | | + | | | | | |
| I | 436 | + | + | | | + | + | + | + | + | + | | + | | + | | | + | + |
| I | 442 | | | + | + | | | | | | | + | | | | + | | | |
| v | 447 | + | + | | | | | | | | | | | + | | | + | | + |
| V5 | 451 | + | + | | | + | + | + | | + | +1 | | | + | | | | | |
| I | 453 | | | | | | + | | + | | | | | | + | | | + | + |
| I | 457 | | | + | + | | | | | | | | | | | + | | | |
| v | 460 | | | | + | | | | | | | | | | | | | | |
| | Subtype | B | B | B | B | B | B | B | B | B | B | G | B | E | B | B | B | B | B |
| | Trans- | HM | HM | HM | HM | HM | HM | HM ? | HM ? | HT | HT | HT | HT | HT | HM | HM | ? | ? | HM |
| | mission | | | | | | | | | | | | | | | | | | |
| | PI/ | PI | PI | PI | PI | PI | PI | PI | PI | PI | PI | PI | PI | PI | TC | TC | TC | TC | TC |
| | TCLA | | | | | | | | | | | | | | | | | | |
| | C.R. | R5 | R5 | R5 | R5 | R5 | R5 | R5 | R5 | R5 | R5 | R5 | R5 | R5 | X4 | X4 | X4 | X4R5 | X4R5 |

+: conserved glycosylation sites
Transmission: HM: homosexual; HT: heterosexual
Primary isolate: PI; TCLA strain: TC
Use of coreceptors: CCR5: R5; CXCR4: X4; double tropism: X4R5
NB:
the amino acid numbering is given relative to the complete sequence.

The inventors then selected the most highly conserved glycosylation sites and positioned these conserved sites on the 3D structure of gp120 (#PDB database: 1G9M) using the PDB-View software (Glaxo-Welcome). The sites buried in the molecule or present in the internal domain of gp120, which domain interacts with gp41 and is therefore barely exposed at the surface of the oligomer, or not at all, were eliminated from the study. Each sequence was analyzed independently. This analysis showed that the conserved sites had an arrangement comparable to the surface of the molecule, Whatever the sequence of the primary isolates.

The inventors then defined “groups” or “glycosylation clusters” which correspond to the grouping together of sites conserved at the surface of gp120 from one primary isolate to another. Since these groups are defined, a list of deglycosylation mutants which takes into account these results was established.

One of the glycosylation groups corresponds to a region located in the proximity of the GPGS motif of the V3 loop (group g14).

Another group (g112/g122) partially recovers the glycosylation sites which define the neutralizing epitope 2G12 (Trkolla A. et al., 1996 (14)).

A third group is juxtaposed with respect to a cavity where amino acids involved in binding to the coreceptors are located. (group 1112).

A fourth group is located within 4 anti-parallel beta-sheets located at the junction of the two main domains of gp120 (bridging-sheet).

A fifth group is present on the “silent” face of gp120 (group 113).

The controls consist either of the completely deglycosylated envelope gene for VI (6) or of a complete deletion of the V1V2 loops.

In parallel, and in order to validate the notion of glycosylation “cluster”, two additional mutants (g123 and g1123) were defined, which correspond to deglycosylations, not within the same cluster, but in each different “cluster” (one mutation per “cluster”)

At least one mutation was effected on the selected sequences obtained from patient 133 (PHI133, accession number AF041126). Each mutation consists of replacement of the asparagine of the potential glycosylation site (NXS/T) with a glutamine, using a site-directed mutagenesis kit according to the manufacturer's instructions (Quickchange Site-directed Mutagenesis Kit, Stratagene). Each mutant was cloned into the expression vector pCI (Promega) and completely sequenced in order to verify the intensity of the sequence after mutagenesis. The mutant plasmids were purified using the Nucleobond PC500 kit (Macherey-Nagel).

FIG. 1 gives a diagrammatic representation of the definition of the clusters and the proximity of each site on the crystallized gp120 molecule.

Table 2 recapitulates the nature and the positioning of each mutation for a given mutant. In table 2, the reference amino acid sequences are the sequence of the deleted, nonmutated Env protein of the HIV-1 isolate 133 and the sequence of the complete, nonmutated Env protein of the HIV-1 isolate 133. The positions where amino acids correspond are given in table 2. The amino acid sequence of the complete, nonmutated Env protein of the HIV-1 isolate 133 is identified in SEQ ID No: 11 and the corresponding (complete) nonmutated nucleotide sequence of the env gene of the HIV-1 isolate 133 is identified in SEQ ID No: 1. The sequences SEQ ID Nos: 2 to 10 and SEQ ID Nos. 12 to 20, which are listed below, correspond respectively to the sequences of the truncated and complete mutated env genes of the HIV-1 isolate 133 and to the sequences of the truncated and complete mutated Env proteins of the HIV-1 isolate 133. The sequence of gp160 is given with reference to SEQ ID No: 11. The sequence of gp120 begins at amino acid 1 and ends at amino acid 498 with reference to SEQ ID No: 11. The sequence of gp140 (which corresponds to gp120 plus the extracellular domains of gp41) begins at amino acid 1 and ends at amino acid 669 with reference to the sequence SEQ ID No: 11.

TABLE 2
characteristics of the various mutants
of the 133 envelope sequence.

| cluster/name of | N->Q mutations of amino acid | N->Q mutations of amino acid | |
|---|---|---|---|
| mutant | no.₍ₐ₎/others | no._{(b)}/others | gp120 regions |
| g1 | 243 | 389 | V4 |
| g11 | 243/231 | 389/377 | V4/V4 |
| g21 | 44 | 190 | C2 (V1V2 stem) |
| g2 | 180-185 | 326-331 | C3 |
| g12 | 180-185/148 | 326-331/294 | C3/V3 |
| g112 | 180-185/148/201-208 | 326-331/294/347-354 | C3/V3/C3 |
| g1112 | 180-185/148/201-208/243 | 326-331/294/347-354/389 | C3/V3/C3/V4 |
| g22 | 180-185/201-208 | 326-331/347-354 | C3/C3 |
| g122 | 180-185/201-208/231 | 326-331/347-354/377 | C3/C3/V4 |
| g3 | 289 | 434 | C4 |
| g13 | 289/109 | 434/255 | C4/C2 |
| g113 | 289/109/123 | 434/255/269 | C4/C2/C2 |
| g23 | 289/123 | 434/269 | C4/C2 |
| g4 | 123 | 269 | C2 |
| g14 | 123/148 | 269/294 | C2/V3 |
| g123 | 289/123/243 | 434/269/389 | C4/C2/V4 |
| (non cluster) | | | |
| g1123 | 289/123/243/180-185 | 434/269/389/326-331 | C4/C2/V4/C3 |
| (non cluster) | | | |
| V1 deglyc. | V1 deglycosylated | | V1 |
| DV1V2 | V1V2 deletion | | V1V2 |
| 133 native | no mutation-native (+control) | | — |
| 133 anti | no mutation-antisense (−control) | | — |

₍ₐ₎according to the deleted 133 sequence [9]
_{(b)}according to the complete 133 sequence.

It is clearly understood that, depending on the primary isolate, the position of the conserved glycosylation sites can vary by 1 to 8 amino acids and the invention encompasses these positional variations.

Table 3 below gives some examples of variation of the glycosylation sites and establishes a recapitulation of all the potential glycosylation sites in the env genes of the primary isolates 133, 146, 153, 159, 160, 374 and 384. The amino acid positions are identical for the deleted sequences according to Kwong et al. 1998. Each line in the table corresponds to a potential site, and the correspondence between the various positions was determined by alignment of the deleted sequences. The glycosylation sites conserved in all the sequences are underlined.

TABLE 3

| Sequence | 133 | 146 | 153 | 159 | 160 | 374 | 384 | mutants |
|---|---|---|---|---|---|---|---|---|
| no aa | 9 | — | — | — | — | — | — | |
| | 44 | 44 | 44 | 44 | 44 | 44 | 44 | |
| | 77 | — | — | 77 | 77 | — | 77 | |
| | — | 81 | 81 | 81 | — | 82 | — | |
| | 88 | 88 | 88 | 88 | 88 | 88 | 88 | |
| | 109 | 109 | 109 | 109 | 109 | 109 | 109 | |
| | 123 | 123 | 123 | 123 | 123 | 123 | 123 | g14 |
| | — | 136 | — | — | 136 | 136 | 136 | |
| | — | 142 | 142 | 142 | 142 | — | 142 | |
| | 148 | 148 | 148 | 148 | 148 | 148 | 148 | g12/g112/ |
| | | | | | | | | g14 |
| | 180 | 178 | — | 178 | 178 | 178 | 178 | |
| | 185 | 185 | 185 | 185 | 185 | 183 | 185 | g12/g112 |
| | 201 | 202 | 202 | 201 | 201 | 201 | 201 | g112 |
| | 208 | — | 208 | 207 | 207 | 207 | 207 | |
| | 231 | — | — | 231 | 231 | — | — | |
| | — | 238 | 238 | 237 | 237 | 238 | 237 | |
| | — | — | 242 | — | 241 | — | 241 | |
| | — | — | 243 | — | — | 243 | — | |
| | 243 | 244 | 247 | 247 | 248 | 247 | 247 | |
| | 253 | — | 253 | 253 | 254 | 255 | 253 | |
| | — | — | 258 | 259 | — | — | — | |
| | 289 | 288 | 295 | 296 | 290 | 290 | 289 | |
| | 301 | 301 | — | — | — | 304 | — | |
| | 306 | 304 | 311 | 311 | 306 | 307 | 305 | |
| | — | — | — | 314 | — | — | — | |

The inventors then studied the antigenic and functional characteristics of each of these mutants and selected the mutants which exhibit the best characteristics for preventive vaccine purposes. The mutants g12, g112 and g14 were selected. The results are given in the examples and figures which follow.

The sequence SEQ ID NO: 2 represents the nucleotide sequence of the g12 mutant (env gene) The sequence SEQ ID NO: 3 represents the truncated nucleotide sequence of the g12 mutant, which encodes gp120.

The sequence SEQ ID NO: 4 represents the truncated nucleotide sequence of the g12 mutant, which encodes gp140.

The sequence SEQ ID NO: 5 represents the nucleotide sequence of the g112 mutant (env gene).

The sequence SEQ ID NO: 6 represents the truncated nucleotide sequence of the g112 mutant, which encodes gp120.

The sequence SEQ ID NO: 7 represents the truncated nucleotide sequence of the g112 mutant, which encodes gp140.

The sequence SEQ ID NO: 8 represents the nucleotide sequence of the g14 mutant (env gene).

The sequence SEQ ID NO: 9 represents the truncated nucleotide sequence of the g14 mutant, which encodes gp120.

The sequence SEQ ID NO: 10 represents the truncated nucleotide sequence of the g14 mutant, which encodes gp140.

The sequence SEQ ID NO: 12 represents the amino acid sequence of the g12 mutant (Env protein).

The sequence SEQ ID NO: 13 represents the gp120 amino acid sequence of the g12 mutant.

The sequence SEQ ID NO: 14 represents the gp140 amino acid sequence of the g12 mutant.

The sequence SEQ ID NO: 15 represents the amino acid sequence of the g112 mutant (Env protein).

The sequence SEQ ID NO: 16 represents the gp120 amino acid sequence of the g112 mutant.

The sequence SEQ ID NO: 17 represents the gp140 amino acid sequence of the g112 mutant.

The sequence SEQ ID NO: 18 represents the amino acid sequence of the g14 mutant (Env protein).

The sequence SEQ ID NO: 19 represents the gp120 amino acid sequence of the g14 mutant.

The sequence SEQ ID NO: 20 represents the gp140 amino acid sequence of the g14 mutant.

The gp120 and gp140 sequences are obtained either by truncating the gene, or by inserting a stop codon and/or a reading frame shift without deletion of the gene. The gp120 and gp140 forms are soluble, and therefore easier to purify and to administer than the complete Env protein.

Thus, a subject of the present invention is a mutated env gene encoding a mutated envelope glycoprotein of the HIV-1 virus, said gene exhibiting, compared to the env gene of a “reference” primary infection primary isolate, at least two mutations at the glycosylation sites conserved from one primary isolate to another, each mutation consisting of replacement of an AAC or AAT codon which encodes an asparagine with a CAG or CAA codon which encodes a glutamine, the two mutations at least being chosen from the following:
(a) at least two mutations in the part of the env gene encoding the C3 region of the env protein,
(b) at least one mutation in the part encoding the C3 region and at least one mutation in the part encoding the V3 region-,
(c) at least one mutation in the part encoding the C2 region and at least one mutation in the part encoding the V3 region chosen from the glycosylation sites at codons 862-864 and 970-972,
(d) at least one mutation in the part encoding the C2 region chosen from the glycosylation sites at codons 667-669, 679-681, 700-702, 763-765 and 844-846, and at least one mutation in the part encoding the V3 region,
it being possible for the position of at least any one of said codons to vary by three to twenty-four nucleotides,
or the mutated env gene consists of any one of the sequences SEQ ID NO: 8, SEQ ID NO: 9 or SEQ ID NO: 10 or the sequences complementary thereto.

One of the advantages of the multiple mutants (at least two mutations) according to the invention lies in the decrease in the probability of having compensatory mutations which would result in a wild-type phenotype again being found.

The preferred mutations according to the invention are as follows.

According to the mutations (a), at least one mutation is effected at codons 976-978, 991-993 of the part encoding the C3 region and at least one mutation is effected at codons 1039-1041 or 1060-1062 of the part encoding the C3 region, it being possible for the position of at least any one of said codons to vary by three to twenty-four nucleotides. Preferably, the gene consists of a sequence chosen from the sequences identified in SEQ ID NO: 21, SEQ ID NO: 22 and SEQ ID NO:23 or the sequences complementary thereto.

According to the mutations (b), at least one mutation is effected at codons 976-978, 991-993, 1039-1041 or 1060-1062 of the part encoding the C3 region and at least one mutation is effected at codon 880-882 of the part encoding the V3 region, it being possible for the position of at least any one of said codons to vary by three to twenty-four nucleotides; according to this variant, the gene comprises at least three mutations, i.e. at least two mutations in the part encoding the C3 region and at least one mutation in the part encoding the V3 region;
preferably, these mutations are effected at codons 976-978, 991-993 and 880-882, it being possible for the position of at least any one of said codons to vary by three to twenty-four nucleotides; advantageously, the sequence of the gene thus mutated consists of a sequence chosen from the sequences identified in SEQ ID NO: 2, SEQ ID NO: 3 and SEQ ID NO: 4 or the sequences complementary thereto, or
at least one mutation is effected at codon 976-978 or 991-993, at least one mutation is effected at codon 880-882, and at least one mutation is effected at codon 1039-1041 or 1060-1062; preferably, the mutations are effected at codons 976-978, 991-993, 880-882, 1039-1041 and 1060-1062; advantageously, the sequence of the gene thus mutated consists of a sequence chosen from the sequences identified in SEQ ID NO: 5, SEQ ID NO: 6 and SEQ ID NO: 7, or the sequences complementary thereto,
it being understood that the position of at least any one of said abovementioned codons can vary by three to twenty-four nucleotides.

According to the mutations (c), at least one mutation is effected at codon 805-807 of the part encoding the C2 region, it being possible for at least any one of said codons to vary by three to twenty-four nucleotides.

According to the mutations (d), at least one mutation is effected at codon 880-882 of the part encoding the V3 region, it being possible for the position of at least any one of said codons to vary by three to twenty-four nucleotides.

The sequence of the mutated gene can also consist of a sequence chosen from the sequences identified in SEQ ID NO: 8, SEQ ID NO: 9 and SEQ ID NO: 10, or the sequences complementary thereto.

The invention also relates to a mutated Env glycoprotein of the HIV-1 virus, which glycoprotein exhibits, compared to a native Env protein of a “reference” primary infection primary isolate, at least two mutations at the glycosylation sites of said reference protein which are conserved from one primary isolate to another, each mutation consisting of replacement of an asparagine with a glutamine, the two mutations at least being chosen from the following:
(a′) at least two mutations in the C3 region of the Env protein,
(b′) at least one mutation in the C3 region and at least one mutation in the V3 region,
(c′) at least one mutation in the C2 region and at least one mutation in the V3 region chosen from the glycosylation sites at amino acid 288 or 324,
(d′) at least one mutation in the C2 region chosen from the glycosylation sites at any one of amino acids 223, 227, 234, 255 and 282, and at least one mutation in the V3 region,
it being possible for the position of said glycosylation sites conserved from one primary infection primary isolate to another to vary by one to eight amino acids,
or the mutated Env glycoprotein consists of any one of the sequences or the mutated env gene consists of any one of the sequences SEQ ID NO: 18, SEQ ID NO: 19 or SEQ ID NO: 20.

The position of the amino acids of the mutated glycosylation sites is given with reference to the sequence of the native Env protein.

The preferred mutations according to the invention are as follows.

According to the mutations (a′), at least one mutation is effected at the glycosylation site at amino acid 326 or 331 and at least one mutation is effected at the glycosylation site at amino acid 347 or 354, it being possible for the position of said glycosylation sites conserved from one primary infection primary isolate to another to vary by one to eight amino acids. Preferably, the glycoprotein consists of a sequence chosen from the sequences identified in SEQ ID NO: 21, SEQ ID NO: 22 and SEQ ID NO: 23.

According to the mutations (b′), at least one mutation is effected at amino acid 326 or 331, and at least one mutation is effected at amino acid 294, it being possible for the position of at least any one of said glycosylation sites conserved from one primary infection primary isolate to another to vary by one to eight amino acids; according to a variant, the glycoprotein comprises at least three mutations, preferably at least two mutations in the C3 region and at least one mutation in the V3 region;
preferably, these mutations are effected at amino acids 326, 331 and 294; advantageously, a mutated Env glycoprotein of the invention consists of a sequence chosen from the sequences identified in SEQ ID NO: 12, SEQ ID NO: 13 and SEQ ID NO: 14; or
at least one mutation is effected at amino acid 326 or 331, at least one mutation is effected at amino acid 347 or 354 and at least one mutation is effected at amino acid 294; advantageously, the mutations are effected at amino acids 326, 331, 294, 347 and 354; better still, a mutated Env glycoprotein of the invention consists of a sequence chosen from the sequences identified in SEQ ID NO: 15, SEQ ID NO: 16 and SEQ ID NO: 17,
it being understood that the position of said glycosylation sites conserved from one primary infection primary isolate to another can vary by one to eight amino acids.

According to the mutations (c′), at least one mutation is effected at amino acid 269 of the C2 region.

According to the mutations (d′), at least one mutation is effected at amino acid 294 of the V3 region.

A mutated Env glycoprotein of the invention can consist of a sequence chosen from the sequences identified in SEQ ID NO: 18, SEQ ID NO: 19 and SEQ ID NO: 20.

A subject of the invention is also a pharmaceutical composition comprising:
(i) at least one mutated gene encoding a mutated gp120 envelope glycoprotein of the HIV-1 virus, said mutated gene being chosen from any one of the genes described above and being placed under the control of regulatory sequences which allow its expression in a host cell,
   a pharmaceutically acceptable vehicle, and optionally, an additional agent which facilitates the penetration of said mutated gene into said cell and/or which makes it possible to target said cell, or
(ii) at least the mutated gene identified in (i) cloned into a recombinant viral vector.

Among the regulatory sequences, mention may be made of the human CMV intermediate/early promoter and the HIV-1 rev gene.

If desired, said host cells or a given cellular compartment of said host cells can be targeted and, to do this, it is possible to couple to the active principle, namely the mutated DNA of the invention, at least one targeting agent. The term “targeting agent” is intended to mean a chemical or biological molecule which makes it possible to target said host cell in vivo. It may, inter alia, be a specific ligand, or an anti-ligand naturally present in the host cell or in the cellular compartment to be targeted, in particular present at the surface of said host cell, and mention may be made., by way of example, of antibodies as ligands and antigens as anti-ligands. Thus, if an antibody is coupled to the active principle and is specific for a surface antigen of the host cell, it will act as an agent for targeting said host cell via the specific formation of an antibody/antigen complex.

The expression “agent which facilitates the penetration of said mutated gene into a host cell” is intended to mean, inter alia, agents such as bupivacaine and cardiotoxin.

The term “pharmaceutically acceptable vehicle” is intended to mean, inter alia, liposomes, virosomes, nanoparticles, microparticles (for example: gold beads), iscoms.

For the recombinant viral vector, mention may be made, by way of example, of MVA (Modified Vaccinia Ankara), alphaviruses, SFV (Semliki Forest Virus), adenoviruses and AAV (Adeno-Associated Virus).

The pharmaceutical compositions defined above are DNA vaccine compositions which are particularly advantageous, in particular compared to the “conventional” vaccine compositions based on recombinant protein. In fact, the use of recombinant proteins for vaccine purposes is a laborious and expensive system, in particular because it requires very important steps for purifying the recombinant antigens. In addition, one of the difficulties encountered is obtaining persistence of the vaccine for a sufficiently long period of time to maintain a good immune memory. Conversely, the method of immunization with DNA, the advantages of which are inherent to the intrinsic properties of DNA, is simple and relatively inexpensive and is carried out simply by intramuscular or intradermal injection. In addition, it should be noted that:
the DNA vaccines are non-infectious/non-replicative,
due to the fact that immunization with DNA is a form of in vivo transfection, the the viral antigen is expressed in the mammalian cells in its native conformation,
as in the case of a viral infection, a broad immune response, both humoral and cellular, is induced, and
in addition, the DNA vaccines can readily be combined due to their physicochemical homogeneity.

However, the invention is not limited to a DNA vaccine composition as defined above, and also relates to a pharmaceutical composition comprising:
at least one mutated gp120 envelope glycoprotein of the HIV-1 virus, said mutated glycoprotein being chosen from any one of the glycoproteins described above,
a pharmaceutically acceptable vehicle and/or excipient and/or adjuvant and/or diluent.

Such a prepared vaccine composition is injectable, i.e. in liquid solution or in suspension. As an option, the preparation may also be emulsified. The antigenic molecule, i.e. the mutated glycoprotein, can be mixed with excipients which are pharmaceutically acceptable and compatible with the active ingredient or principle. Examples of favorable excipients are water, a saline solution, dextrose, glycerol, ethanol or equivalents, and combinations thereof. If desired, the vaccine can contain minor amounts of auxiliary substances such as wetting agents or emulsifiers, pH-buffering agents or adjuvants such as aluminum hydroxide, muramyl dipeptide or variations thereof, cationic polymers, nanoparticles of cationic polymers or variants thereof (poly(lactic acid), poly(lactic acid)-coglycoside).

The vaccine is administered conventionally by injection, for example intramuscular injection.

The term “pharmaceutically acceptable vehicle” is intended to mean the carriers and vehicles which can be administered to humans or to animals, as described, for example, in Remington's Pharmaceutical Sciences 16^{th} ed., Mack Publishing Co. The pharmaceutically acceptable vehicle is preferably isotonic or hypotonic or is slightly hypertonic and has a relatively low ionic strength. The definitions of the pharmaceutically acceptable excipients and adjuvants are also given in Remington's Pharmaceutical Sciences mentioned above.

The present invention also relates to a method for inducing an improved humoral and/or cellular response against the HIV-1 virus in a mammalian animal, according to which:
an amount of a DNA vaccine composition, comprising at least the mutated gene which encodes a mutated protein of the invention as defined above, which is sufficient to be effective is administered to a mammalian animal by intramuscular or intradermal injection, or
an amount of a vaccine composition, comprising at least a mutated protein of the invention as defined above, which is sufficient to be effective is administered to a mammalian animal by intramuscular or intradermal injection.

In the case of DNA vaccines, the concentration of nucleic acid in the composition used for administration in vivo is approximately 100 μg/ml to 10 mg/ml, preferably 1 mg/ml.

The amount of protein administered depends on whether or not an adjuvant is added, but will generally be between 10 and 50 μg/ml of protein.

The vaccine is administered at a given dose in one or more injection(s) intramuscularly or intradermally, followed by a booster or boosters, if necessary. The immunizing effect of the vaccine is monitored by measuring the titer of anti-HIV-1 antibodies in the individual or the animal immunized.

The administration of a nucleic acid or nucleic acids or of a protein or proteins of interest, or of a fragment or fragments thereof, alone or in combination, is used for prophylaxis and/or therapy. When one or more nucleic acid(s) or fragments thereof or one or more protein(s) or fragments thereof are administered, their characteristic is to not exhibit the virulence of HIV-1 but to have the property of inducing a humoral and/or cellular immune response in the individual or in the animal to whom or to which they are administered. When a protein or proteins is or are involved, said protein(s) can be obtained by synthetic techniques or genetic recombination techniques or by modification of natural protein(s) by chemical or physical treatments.

The protein(s), or fragments thereof, which is (are) a candidate or candidates for the formation of a vaccine identified and analyzed in a functional assay in order to be sure that they have lost their toxicity and to verify their immunogenicity (i) by carrying out an in vitro assay for proliferation of CD4+ T lymphocytes specific for the administered antigen (T cell assay) or an in vitro assay for cytotoxicity of the CD8+ lymphocytes specific for the administered antigen, and (ii) by measuring, inter alia, the titer of circulating antibodies directed against the natural protein and their ability to neutralize primary isolates. These modified forms are used to immunize humans by standardized procedures with suitable adjuvants.

The invention also relates to:
a method for obtaining antibodies, according to which a mammalian animal, such as a mouse, rat or monkey, is immunized with at least one mutated gene encoding a mutated envelope glycoprotein of the HIV-1 virus, said mutated gene being chosen from any one of the genes described above and being placed under the control of regulatory sequences which allow its expression in vivo, and the antibodies which can be obtained by this method, and
a method for obtaining antibodies, according to which a mammalian animal, such as a mouse, rat or monkey, is immunized with at least one mutated envelope glycoprotein of the HIV-1 virus, said mutated glycoprotein being chosen from any one of the glycoproteins described above, and the antibodies which can be obtained by this method.

The term “antibodies” is intended to mean polyclonal antibodies, monoclonal antibodies, transmembrane antibodies and humanized antibodies, or fragments of said antibodies. The production of polyclonal and monoclonal antibodies is part of the general knowledge of those skilled in the art. By way of reference, mention may be made of G. Köhler and C. Milstein (1975): Continuous culture of fused cells secreting antibody of predefined specificity, Nature 256:495-497 and G. Galfre et al. (1977) Nature, 266: 522-550 for the production of monoclonal antibodies, and A. Roda, G. F. Bolelli: Production of high-titer antibody to bile acids, Journal of Steroid Biochemistry, Vol. 13, pp. 449-454 (1980) for the production of polyclonal antibodies. For the production of monoclonal antibodies, the immunogen can be coupled to Keyhole Limpet Hemocyanin (KLH peptide) as carrier for the immunization or to serum albumin (SA peptide). The animals are given an injection of the immunogen using Freund's complete adjuvant. The sera and the hybridoma culture supernatants derived from the immunized animals are analyzed for their specificity and their selectivity using conventional techniques, such as, for example, ELISA or Western blotting assays. The hybridomas producing the most specific and most sensitive antibodies are selected. Monoclonal antibodies can also be produced in vitro by cell culture of the hybridomas produced or by recovery of ascites fluid, after intraperitoneal injection of the hybridomas into the mouse. Whatever the method of production, as supernatant or as ascites, the antibodies are then purified. The purification methods used are essentially ion exchange gel filtration and exclusion chromatography or affinity chromatography (protein A or G). A sufficient number of antibodies are screened, in funtional assays, to identify the most effective antibodies. The in vitro production of antibodies, of antibody fragments or of antibody derivatives, such as chimeric antibodies produced by genetic engineering, is well known to those skilled in the art.

The term “transmembrane antibody” is intended to mean an antibody in which at least the functional region capable of recognizing and of binding to its specific antigen is expressed at the surface of the target cells so as to allow said recognition and binding. More particularly, the antibodies according to the present invention consist of fusion polypeptides comprising the amino acids defining said functional region and a sequence of amino acids (transmembrane polypeptide) allowing anchoring within the membrane double lipid layer of the target cell or at the outer surface of this bilayer. The nucleic acid sequences encoding many transmembrane polypeptides are described in the literature.

“Humanized” forms of non-human antibodies, for example murine antibodies, are chimeric antibodies which comprise a minimum sequence derived from a non-human immunoglobulin. For the most part, humanized antibodies are human immunoglobulins (receptor antibody) in which residues of a hypervariable region of the receptor are replaced with residues of a hypervariable region of a non-human donor species (donor antibody), such as mouse, rat, rabbit or a non-human primate, having the desired specificity, affinity and capacity. In certain cases, the residues (FR) of the Fv region of the human immunoglobulin are replaced with corresponding non-human residues. In addition, human antibodies can comprise residues which are not found in the recipient antibody or in the donor antibody. These modifications are performed in order to improve the performance levels of the antibody. In general, the humanized antibody will comprise at least, and preferably, two variable domains, in which all or virtually all of the hypervariable loops correspond to a non-human immunoglobulin and all or virtually all of the FR regions will be those of a human immunoglobulin. The humanized antibodies may also optionally comprise at least part of a constant (Fc) region of an immunoglobulin, such as a human immunoglobulin (Jones et al., Nature 321: 522-525 (1986); Reichmann et al., Nature 332: 323-329 (1988); and Presta et al., Curr. Op. Struct. Biol. 2: 593-596 (1992)).

The term “antibody fragment” is intended to mean the F(ab)₂, Fab, Fab′ and sFv fragments (Blazar et al., 1997, Journal of Immunology 159: 5821-5833 and Bird et al., 1988, Science 242: 423-426) of a native antibody, and the term “derivative” is intended to mean, inter alia, a chimeric derivative of a native antibody (see, for example, Arakawa et al., 1996, J. Biochem 120: 657-662 and Chaudray et al., 1989, Nature 339: 394-397).

These antibodies can be incorporated into a pharmaceutical composition, in particular when they are neutralizing antibodies, in order to respond to a viral infection with HIV-1, and the invention encompasses such a pharmaceutical composition.

Finally, the invention relates to a method for evaluating a therapeutic agent, according to which at least one mutated gene encoding a mutated envelope glycoprotein of the HIV-1 virus as described above is administered to an animal, and the following are carried out:
measurement of the titer of antibodies specific for the mutated gp120, and/or
measurement of the titer of neutralizing antibodies specific for the mutated or native glycoprotein which will induce a decrease in the infectivity of the virus, and/or
measurement of the cellular immune response induced against the mutated gp120, for example using an assay for in vitro activation of T “helper” lymphocyte cells specific for the mutated glycoprotein.

For a better understanding of the experimental section which follows, it is advisable to again briefly go over the mechanism of infection of HIV-1. During the infection, gp120 binds to the CD4 receptor. Subsequent to this interaction, the gp120 undergoes a structural rearrangement which releases “CD4-induced” epitopes which allow binding of the coreceptor. The binding of CD4 and then that of the coreceptor are events which are prerequisites for the anchoring of the gp41 fusion peptide in the cell membrane and therefore the infection of the cell.

### FIGURES

FIG. 1 gives a diagrammatic representation of the definition of the clusters and the proximity of each site on the crystallized gp120 molecule.

FIG. 2 represents the detection of gp160 □ and of gp120 □ in each cell lysate for each of the mutants produced and for the controls.

FIG. 3A represents the antigenicity assayed by ELISA in the cell lysates with respect to a pool of HIV-positive sera, and FIG. 3B represents the antigenicity assayed by ELISA in the culture supernatants with respect to the pool of HIV-positive sera.

FIG. 4A represents the amount of CD4s detected in the cell lysates., and FIG. 4B represent the amount of CD4s detected in the culture supernatants.

FIG. 5A represents the recognition of the F105 epitope assayed by ELISA in the culture supernatants, and FIG. 5B represents the recognition of the F105 epitope assayed by ELISA in the cell lysates.

FIG. 6A represents the recognition of the CG10 epitope in the presence of CD4, assayed by ELISA, in the cell lysates, and FIG. 6B represents the recognition of the CG10 epitope alone (▪) and in the presence of CD4 (□), assayed by ELISA, in the culture supernatants.

FIG. 7A represents the recognition of the 17b epitope alone (□) and in the presence of CD4 (□), assayed by ELISA, in the cell lysates, and FIG. 7B represents the recognition of the 17b epitope alone (□) and in the presence of CD4 (□), assayed by ELISA, in the culture supernatants.

FIG. 8 illustrates the neutralization of the FR virus (X4 primary virus) on P4PCCR5 Hela cells with the serum from mice immunized with various constructs (example 4.

FIG. 9 illustrates the infection of GHOST-CCR5 and GHOST-CXCR4 cells with pseudoparticles bearing the mutated glycoproteins identified in table 2.

FIG. 10 illustrates the neutralization of the pseudoparticles bearing the mutated glycoproteins with a neutralizing CRI/LY human serum.

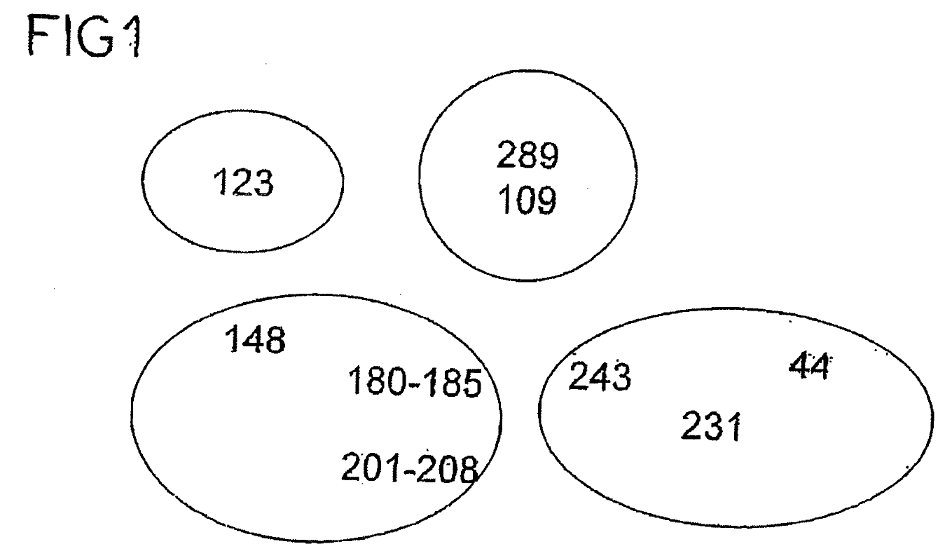

### EXAMPLES

### Example 1

### Expression and Cleavage

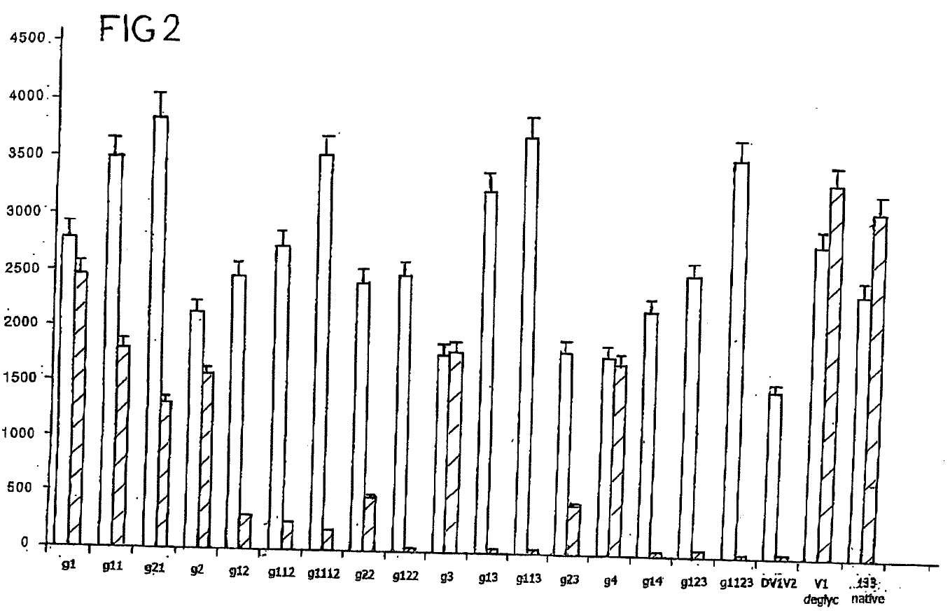

293T cells (kidney epithelial cells) are cotransfected with each of the constructs PCI-env and the expression plasmid PCI-rev using the Lipofectamine-Plus kit (Gibco-BRL) according to the manufacturer's instructions. Rev is a regulatory protein of HIV-1 which plays an important role in expression of the envelope protein. Forty eight hours after transfection, the culture supernatant and the cells are recovered and assayed by Western blotting with an anti-gp120 sheep polyclonal antibody (Biogenesis) diluted to 1/5000. For this, the samples are deposited on a denaturing 8% acrylamide gel. After separation, the samples are transferred onto a PVDF membrane (ImmobilonP, Amersham) according to the manufacturer's instructions. For the lysates, the samples were standardized with an anti-actin monoclonal antibody diluted to 1/5000 (A4700, Sigma) and an anti-Rev monoclonal antibody 2D4D10 diluted to 1/5000 (bioMérieux) For the supernatants, the standardization was carried out according to the amount of serum albumin in the culture medium. The blots are revealed using the ECF kit (Amersham) and the bands revealed are quantified on a Typhoon 8600 using the ImageQuant software. All the constructs express a protein which reacts with the anti-gp120 polyclonal. The migration of the mutant proteins is affected by the loss of one or more glycosylations. The decrease in gp120 detected in the cell lysate is associated with the increase in the number of mutations (FIG. 2). Sometimes, the specific band is completely absent (mutants g1112, g122, g13, g113, g14, g123, g1123 and DV1V2). It is also absent from the corresponding culture supernatant: it may be that the degree of cleavage is affected. Moulard (15) described this phenomenon, which he associates with poor translocation in the ER and/or deficient folding preventing cleavage site accessibility. Conversely, for the g14 mutant, a large amount of gp120 is detected in the supernatant (approximately 8 times more than the native protein, at standardized protein amount) whereas said gp120 is not detected in the cell lysate. It therefore appears that the mutations present on this clone affect the interactions linking the envelope subunits to one another and not the degree of cleavage of gp160. The degree of cleavage for the single mutants is generally comparable to that observed for the native envelope (except for the g21 mutant). It is interesting to note that, by comparison, the mutant corresponding to complete deglycosylation of the V1 loop appears to be relatively unaffected by the loss of three glycosylations.

### Example 2

### Ability of the Mutants to Form Syncytia in vitro

Forty-eight hours after transfection, the 293T cells which express the various mutants are split 1/10 and brought into contact with GHOST-CCR5 or GHOST-CxCR4 cells at 50% density.

After coculturing for 16 hours, the cells are fixed with 0.5% glutaraldehyde for 15 minutes and the nuclei and the cytoplasms are then stained with May-Grünwald/Giemsa. The observation and the counting of the syncytia formed are assessed by observation under an inverted optical microscope.

For the GHOST-CCR5 cells, the ability to induce syncytia is decreased when the number of mutations is increased. It is zero for the clones which are not cleaved (g113, g1123 and perhaps g1112). This result confirms the previous results and gives an indication regarding maintenance of the functionality of the various envelopes after mutation. The constructs do not induce the formation of syncytia on CXCR4 indicator cells, as can be demonstrated by Pollakis (16) after partial deglycosylation of the V3 loop of gp160 ADA.

The results are given in table 4.

TABLE 4
Ability of the envelope mutants to form
syncytia in vitro.

| Constructs | G1 | g11 | g21 | g2 | g12 | g112 | G1112 | G22 | g122 | g3 | g13 | g113 | g23 | g4 | g14 |
|---|---|---|---|---|---|---|---|---|---|---|---|---|---|---|---|
| FS CCR5 | ++ | ++ | + | ++ | + | + | ? | ++ | + | ++ | + | − | ++ | ++ | + |
| FS CXCR4 | − | − | − | − | − | − | − | − | − | − | − | − | − | − | − |

| constructs | G123 | g1123 | DV1V2 | dgV1 | native | anti | HxB2 |
|---|---|---|---|---|---|---|---|
| FS CCR5 | + | − | − | ++ | ++ | − | − |
| FS CXCR4 | − | − | − | − | − | − | + |

FS CCR5: formation of syncytia CCR5 FS CCR4: formation of syncytia CCR4

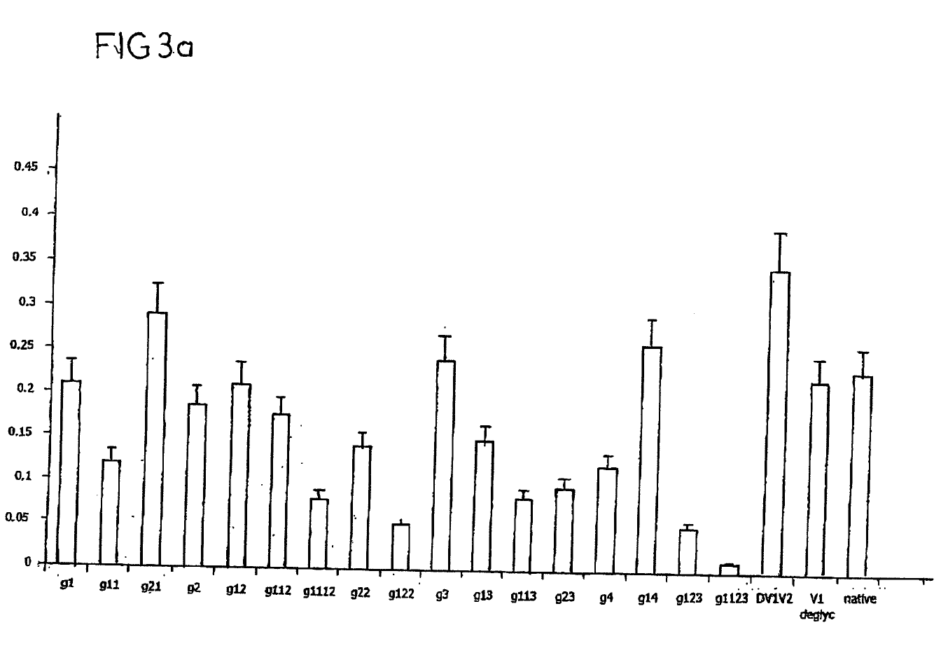

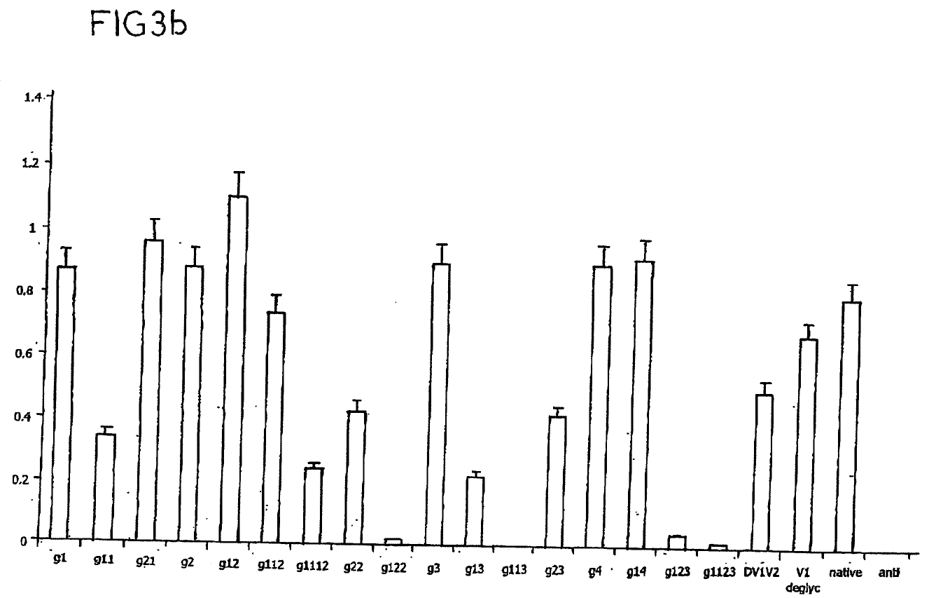

Antigenicity with Respect to a Pool of HIV-Positive Sera (cf. FIG. 3).

The lysates and the supernatants were assayed by capture ELISA with a pool of HIV+ sera originating from the Croix-Rousse Hospital in Lyon. This pool of sera consists of two subtype B samples and one subtype C sample. All the ELISA results correspond to the mean of two experiments carried out in duplicate. The assay format is as follows:
Capture antibody: D7324 (directed against the alternate cleavage site located in the C5 region; Aalto) at 1 μg/ml in 1×PBS or 12G10B11 (directed against gp41, bioMérieux) at 5 μg/ml in 1×PBS; incubation overnight at 6° C.
Saturation with 1×PBS-0.1% Tween-3% powdered skimmed milk; incubation for 1 hour at 22° C.
Lysates diluted to 1/10 in PBS-1% NP40-20 μg/ml BSA, or pure supernatants; incubation for 2 hours at 22° C.
Pool of HIV+ sera diluted to 1/100 in 1×PBS-0.1% Tween-20 μg/ml BSA; incubation for 1 hour at 22° C.—
Peroxidase-coupled anti-Fc human IgG conjugate (Jackson) diluted to 1/5000 in 1×PBS-0.1% Tween-20 ug/ml BSA; incubation for 30 minutes at 22° C.
Revelation with a mixture of 20 mg OPD (Pierce) and 10 ml of Color2 (BioMérieux); incubation for 15 minutes at 22° C. before reading at 492 nm on a plate reader (Biorad).

Four washes with 1×PBS-0.1% Tween are carried out between each incubation, with a Biorad plate washer.

The supernatants are captured with the D7324 antibody; the lysates are captured either with D7324 or with 12G11B10. The latter was mainly used since the D7324 epitope is relatively unexposed on non-cleaved gp160 molecules and the detected signal then depends on the degree of cleavage of the various mutants.

The results show that the loss of antigenicity correlates with the number of mutations. The results on the lysates (12G11B10 coating) and on the supernatants coincide and confirm the absence of gp120 in the supernatants for the g113 and g1123 mutants.

### Example 3

### Recognition

Recognition of the 2G12 Epitope.

2G12 is a human monoclonal antibody obtained by electrofusion of HIV+ PBL and CB-F7 cells (17). It has a broad-spectrum neutralizing capacity on primary isolates and some TCLA viruses. The epitope recognized by this antibody is discontinuous and covers mainly the V3 and V4 regions and also some sugars present in these regions. The 2G12 recognition was assayed by ELISA on the cell lysates and on the supernatants of the various mutants. The assay format is the same as previously, except for the detection antibody, which is 2G12 (1 μg/ml). No positive signal is detected, even for the 133-native protein. This result confirms results obtained by RIPA and by ELISA by the team of D. Brand (Tours) on the 133 envelope. Parren, in 1998, described a primary isolate resistant to the three neutralizing antibodies 2G12, 2F5 and B12. His results indicate that this resistance is probably due to an overall change in the oligomeric structure of the envelope.

Recognition of the CD4-Binding Site.

The envelope glycoprotein has the ability to bind CD4 with high affinity. This binding capacity is crucial in the infection process.
Binding of soluble CD4 (CD4s). The following ELISA format was used:
Coating D7324 (supernatant) or 12G11B10 (lysate).
Saturation.
Lysate diluted to 1/10 or pure supernatant.
CD4s (NIAID) diluted to 1 μg/ml in 1×PBS-0.1% Tween-20 μg/ml BSA; incubation for 2 hours at 22° C.
Biotinylated anti-CD4 polyclonal diluted to 1 μg/ml in 1×PBS-0.1% Tween-20 μg/ml BSA; incubation for 1 hour at 22° C.
Streptavidin-peroxidase (Jackson) diluted to 1/10 000 in 1×PBS-0.1% Tween-20 μg/ml BSA; incubation for 30 minutes at 22° C.
Revelation with a mixture of 20 mg OPD (Pierce) and 10 ml of Color2 (bioMérieux).
Reading at 492 nm.

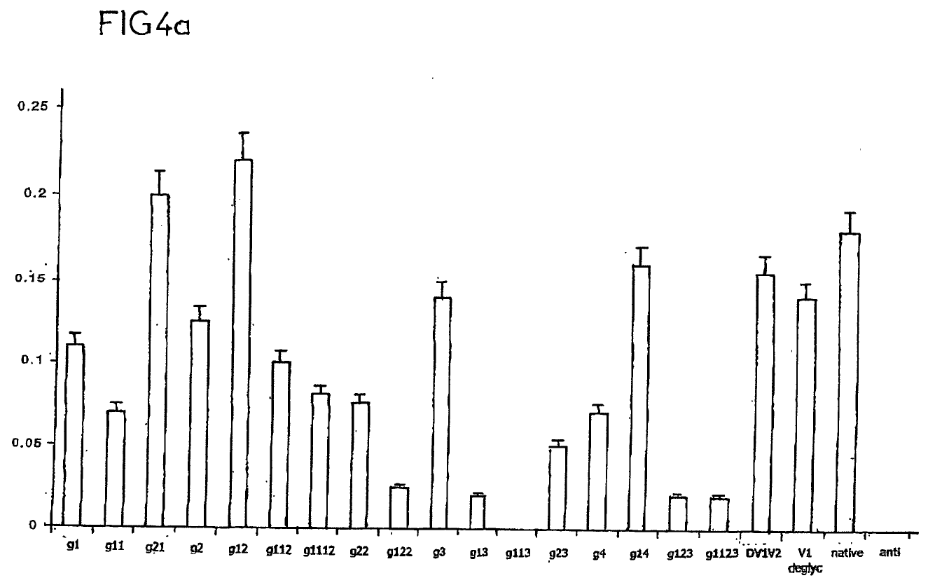

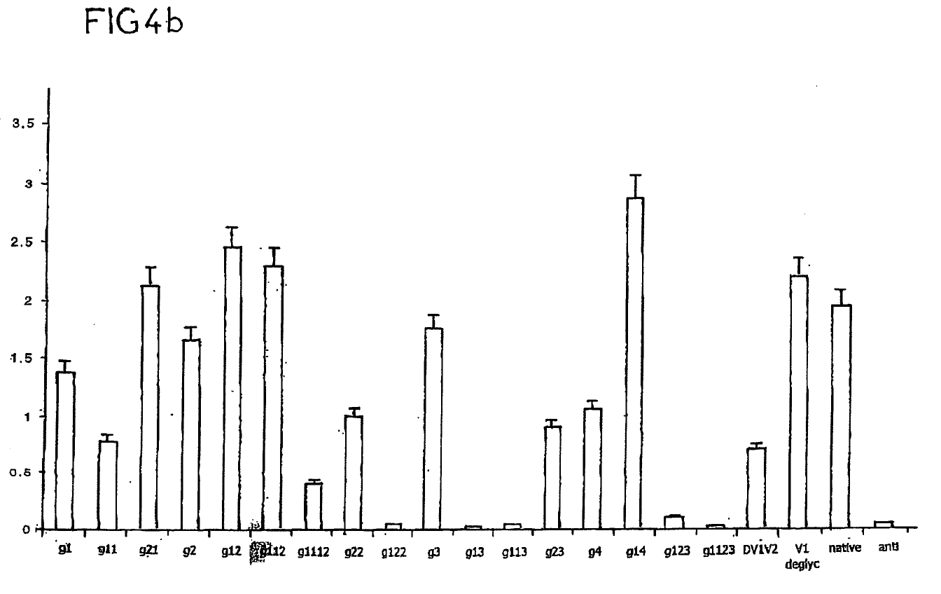

The amount of CD4s detected varies in the same way on the lysates and on the supernatants (FIG. 4). The intensity of the signal depends on the number of deglycosylated sites, except for the g12 and g14 mutants which correspond to double mutations. The clones corresponding to envelope proteins for which the antigenicity or the degree of cleavage is reduced have a low signal (g122, g13, g113, g123 and g1123).

Recognition of the IgG1b12 Epitope.

This human monoclonal antibody is described as having a considerable neutralizing activity both on primary isolates (clade A-D) and on laboratory strains. The epitope is discontinuous and covers the CD4-binding site. The recognition preferentially takes place on the oligomers at the surface of the infected cells or of the viruses, at the expense of the “debris” which prevents an effective neutralizing response.

No positive signal was noted whatever the clones tested. This is in agreement with the preliminary results of D. Brand on the 133 envelope. IgG1b12 is described as being very sensitive to substitutions in the V1 and V2 loops. Some amino acids in the C2 and C3 regions are also described as playing a crucial role in recognition of the b12 epitope (18). One of the particularities of the 133 envelope is that it has an atypical protein sequence compared to the other envelope proteins of the same subtype.

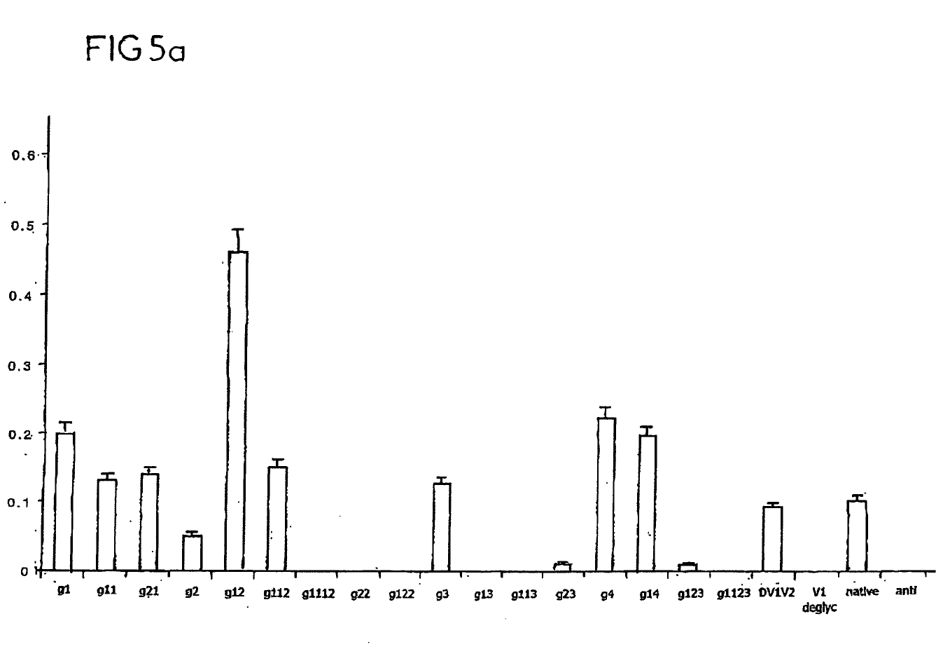

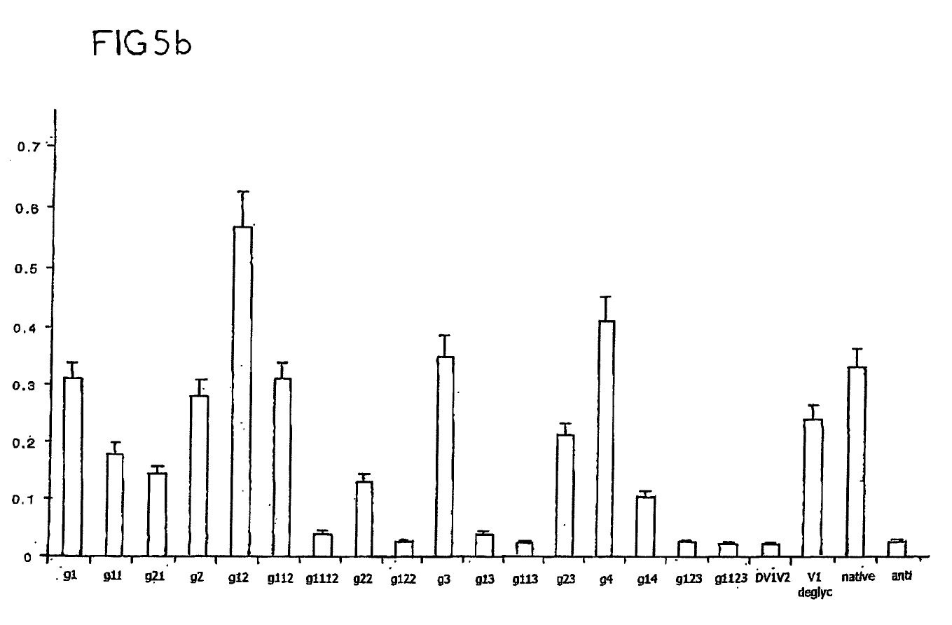

Recognition of the F105 Epitope (cf. FIG. 5).

The human monoclonal antibody F105 binds in the vicinity of the CD4-binding site. CD4s can inhibit its binding. The F105 antibody neutralizes the TCLA strains. Some authors have described neutralization for some primary isolates (19), but the results are very controversial.

The binding of this antibody was tested on the lysates and the supernatants. The assay format is the same as that used with 2G12. The F105 antibody (NIBSC) is used at a concentration of 1 μg/ml. A considerable signal is noted for the g12 clone (up to 4.5 times the signal of the native protein at equivalent protein amount). Mutation no. 294 present on g12 (but not on g2) is located in the V3 loop; it is known that the binding of F105 to V3-deleted glycoproteins is improved. Other mutations appear to have a crucial role in recognition of the protein by the antibody. This is the case for mutation no. 255, which is present in the g13 and g113 clones. It is located in a peptide of C2 described as being important in evading neutralization by F105 (20). There is no signal for the g1112 mutants and those of the g22/g122 series.

Recognition of Induced CD4 Epitopes

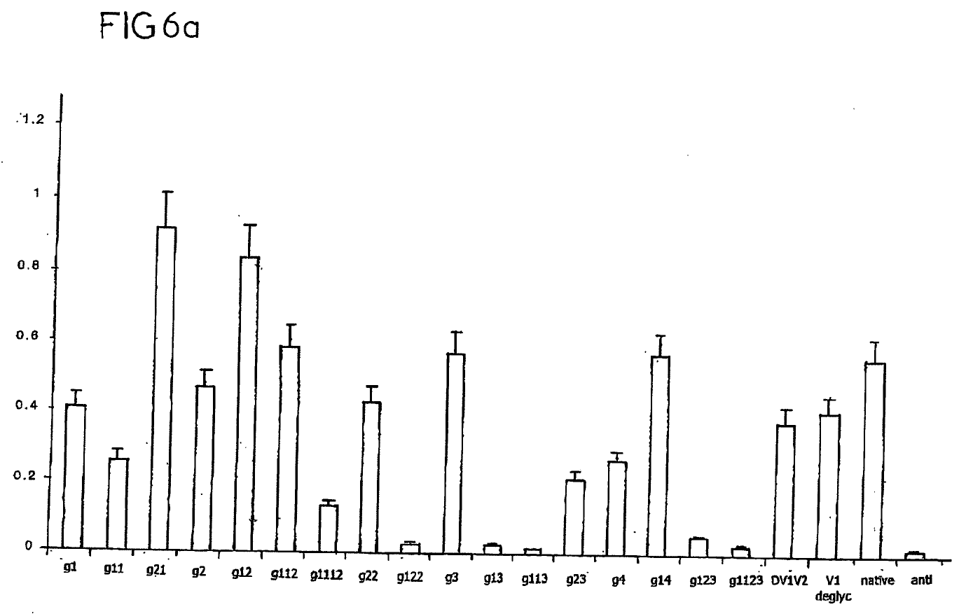

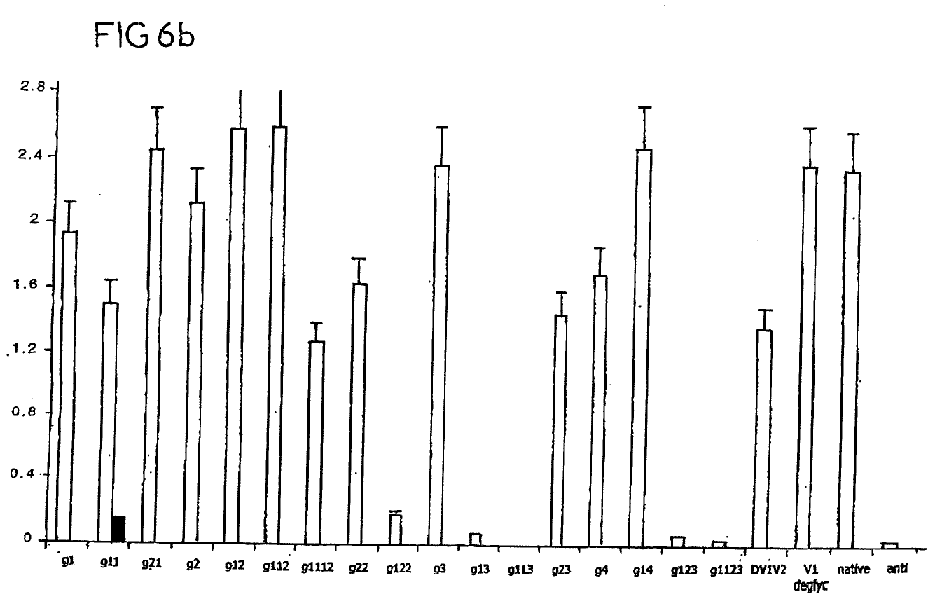

The binding of CD4 induces conformational changes and allows the de novo formation of epitopes, the emergence of which conditions the binding of the coreceptor.
Recognition of the CG10 epitope (FIG. 6). CG10 is a murine monoclonal antibody capable of neutralizing certain TCLA strains. It binds in the vicinity of the “bridging-sheet” in competition with 17b. Its binding is strictly CD4-dependent (21).

The ELISA used has the following format:

Coating: D7324 (supernatants) or 12G11B10 (lysates).

Saturation.

Lysates diluted to 1/10 or pure supernatants.

Addition (or no addition) of CD4s at 1 μg/ml diluted in 1×PBS-0.1% Tween-20 μg/ml BSA; incubation for 2 hours at 22° C.

CG10 (gift from Francisco Veas, UMR 5087, Montpellier) at 1 μg/ml diluted in 1×PBS-0.1% Tween-20 μg/ml BSA; incubation for 1 hour at 22° C.

Peroxidase-coupled anti-mouse IgG conjugate (Jackson) diluted to 1/5000 in 1×PBS-0.1% Tween-20 μg/ml BSA.

Revelation: 20 mg OPD (Pierce) and 10 ml of Color2 (bioMérieux).

Reading at 492 nm.

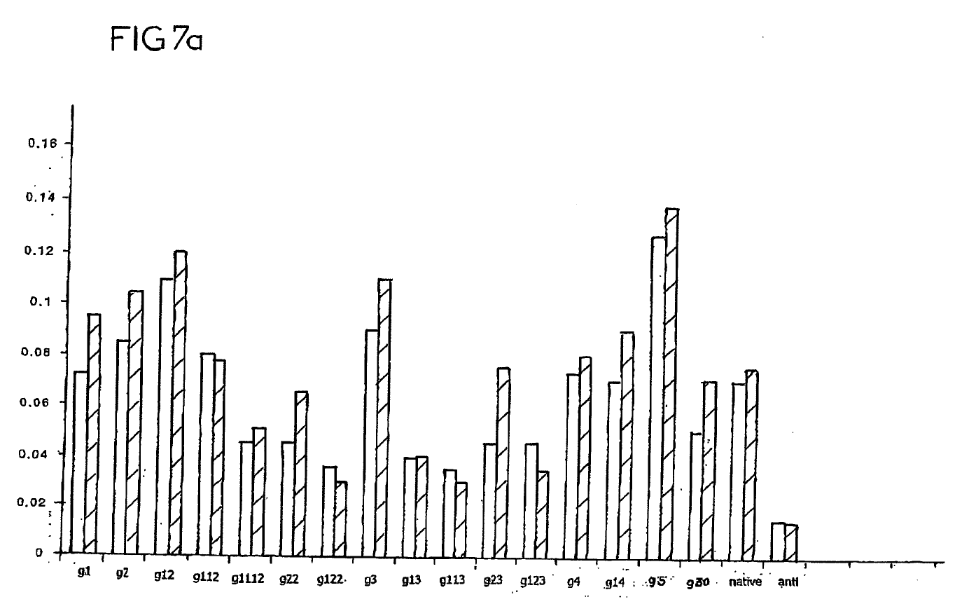

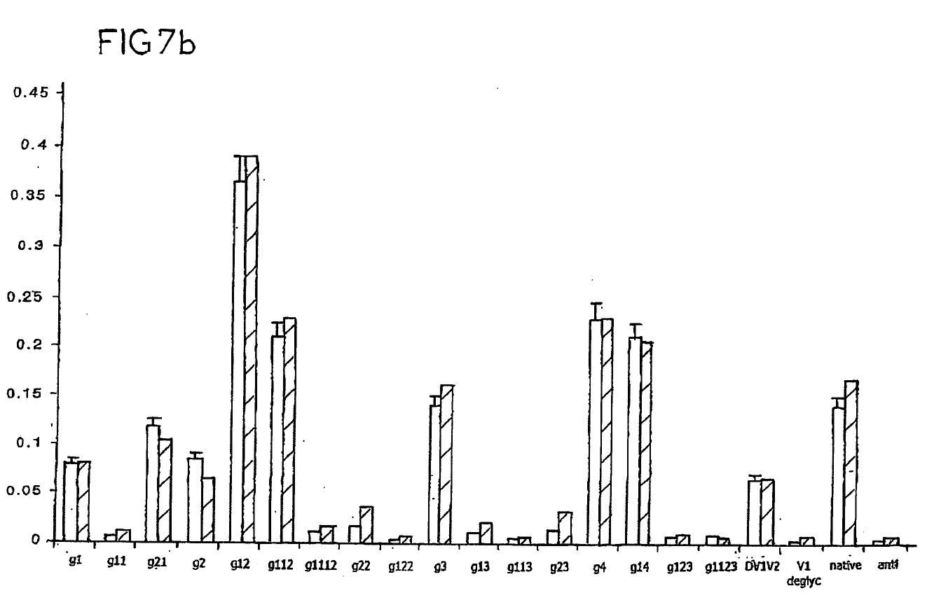

The results by ELISA show correct recognition of the CG10 epitope in the presence of CD4s and for the majority of the envelope proteins, with the exception of the g122, g13, g113, g123 and g1123 mutants. The signal correlates well with the amount of CD4s binding (cf. FIG. 5). The use of CG10 alone results in a lack of signal.
Recognition of the 17b epitope (cf. FIG. 7). 17b is a human monoclonal antibody directed against a CD4-induced epitope. It has a limited neutralizing activity both for primary isolates and TCLA strains. While the exposure of the epitope does not strictly depend on the binding of CD4, it is nevertheless improved by its presence. The ELISA format used is the same as for CG10. The 17b antibody is used at a concentration of 1 μg/ml. The detection is carried out with a peroxidase-coupled anti-human IgG Fc antibody diluted to 1/5000 (Jackson). In the experiment, it is observed that some mutants exhibit correct recognition of this antibody, and in particular the g12/g112, g3 and g4/g14 mutants. For g12, the signal is approximately 3 times greater than that recorded for the native protein at equivalent protein amount. The mutations present in g12/g112 concern the V3 loop and the beginning of the C3 region. According to Wyatt (11), V2 and V3 are thought to partially cover the 17b epitope and it is the binding of CD4 which would allow a withdrawal of the V2 loop. It is possible to imagine that deglycosylation of the V3 loop (NB: the V3 loop of the 133 sequence has a single potential glycosylation site) and of the sites located at its base would allow both a partial demasking of the V3 loop and an increase in its flexibility, which might consequently result in better accessibility of the 17b epitope.

The experiments detailed above show the advantage of the g12, g112 and g14 mutants with regard to their antigenic and functional characteristics.

The immunogenicity of the g12 and g112 mutants (good affinity for F105 and for 17b) should be good. The mutants of the g4/g14 series are advantageous since they have good reactivity with respect to the various antibodies tested.

### Example 4

### Selection of a Panel of Mutants and Evaluation of the Neutralizing Capacity of the Antibodies Generated after Immunization of Balb/C Mice

The following panel of mutants: g12, g112, g14 was chosen according to the ELISA results. The g13 mutant was also evaluated due to the clustered positioning of the mutated sites on the “silent” face of the external domain of gp120. The g123 mutant (non-cluster mutant), and also the gene of the wild-type envelope, were used as references.

Each envelope gene was subcloned into a vector pCi-Rev, which derives from the vector pCi-Neo (Promega) by replacement of the neomycin gene with the rev gene, which is then under the control of the SV40 promoter. Each construct was prepared with an endotoxin-free plasmid extraction kit (Macherey-Nagel PC2000 EF).

The immunizations were carried out in the following way: for each construct, 5 female Balb/C mice were immunized by biolistics (GeneGun, Biorad) using gold particles pre-coated with 4 μg of plasmid to be injected. Five successive injections were given on D0, D14, D28, D54 and D68. On D40 and D80, blood samples were taken from the eye of the animals. These samples were used to test the increase in antibodies directed against the envelope after the various immunizations. To do this, ELISA assays were carried out: gp160 envelope protein (ABL) diluted to 1 μg/ml was adsorbed onto 96-well plates (Nunc). After saturation of the nonspecific sites, the sera from the immunized mice on days D0, D40 and D80 are used at dilutions ranging from 1/100 to 1/800, and then revealed with a horseradish peroxidase-coupled antibody directed against mouse antibodies (Jackson). An OD read at greater than 0.35 is considered to be significant. Table 5 below recapitulates the positive mice obtained for each construct.

TABLE 5

| | mice “positive” |
|---|---|
| constructs | by ELISA |
| pCI-Rev-g12 | S3, S4 |
| pCI-Rev-g112 | S1, S2 |
| pCI-Rev-g13 | S5 |
| pCI-Rev-g14 | S3, S4 |
| pCI-Rev-g123 | S2, S4, S5 |
| pCI-Rev-env wild-type | S1, S2, S3, S4 |

The sera from these mice were used in two neutralization assays described below:

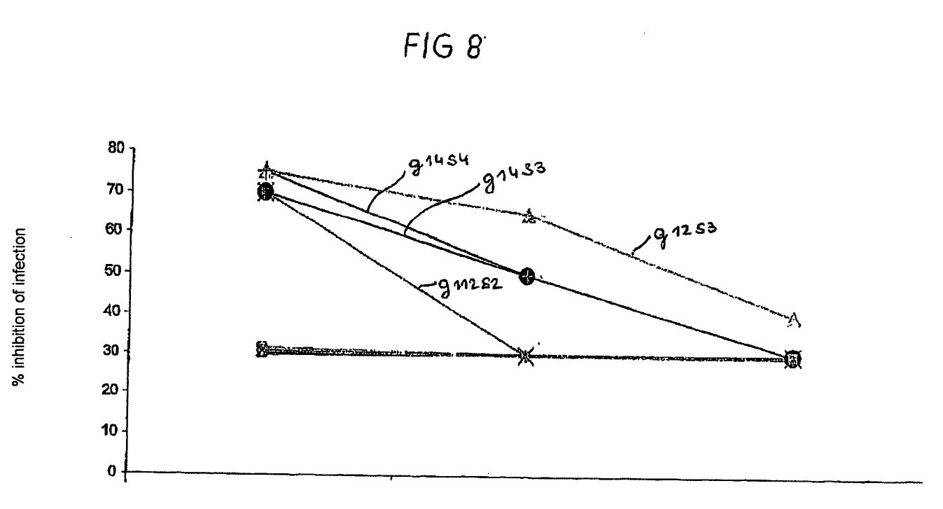

a—The FR virus (X4 primary virus) was brought into contact with serial dilutions (at 1/10, 1/20 and 1/40) of the various mouse sera for 1 h at 37° C. in DMEM medium (Euromedex) and then added to 96-well plates in which P4PCCR5 Hela cells had previously been placed in culture. These cells possess the CD4 receptor and the CCR5 and CXCR4 coreceptors. They have the β-galactosidase gene, under the control of a retroviral LTR. After coculturing for 1 h, 200 μl of DMEM medium containing 5% of fetal calf serum are added. The cels are then incubated for 48 h at 37° C. At the end of this 24 h period, the β-galactosidase expression is revealed with a solution of X-gal at 400 μg/ml in 0.2 M potassium ferricyanide/0.2 M potassium ferrocyanide/2 M MgCl₂. The percentage neutralization corresponds to the percentage of uninfected cells for which β-galactosidase synthesis has not been induced. Assays were carried out in parallel with virus but without mouse serum (infection control) or with nonimmunized mouse serum (neutralization background noise control, Tneg). The results obtained are summarized in table 6 below and FIG. 8.

TABLE 6

| | | | |
|---|---|---|---|
| TnegS1 | 30 | 30 | 30 |
| TnegS2 | 30 | 30 | 30 |
| g12-S3 | 75 | 65 | 40 |
| g12-S4 | 70 | 30 | 30 |
| g13-S5 | 30.5 | 30 | 30 |
| g14-S3 | 70 | 50 | 30 |
| g14-S4 | 75 | 50 | 30 |
| g112-S1 | 30.2 | 30 | 30 |
| g112-S2 | 29.7 | 30 | 30 |
| g123-S3 | 30.5 | 30 | 30 |
| g123-S4 | 31.5 | 30 | 30 |
| g123-S5 | 31 | 30 | 30 |
| wt-S1 | 30.7 | 30 | 30 |
| wt-S2 | 30.5 | 30 | 29.7 |

It is observed that, for the majority of the sera, there is no neutralizing response which differentiates from the background noise (approximately 30% neutralization). However, for the constructs pCI-rev-g12 and pCI-rev-g14, the mice positive by ELISA (respectively S3/S4 and S3/S4) give a significant neutralizing response at the 1/10 dilution (70% neutralization). This percentage is lower for a dilution at 1/20 and returns to the value of the background noise for a dilution at 1/40, except for the S3 mouse immunized with pCI-rev-g12, for which the value is slightly higher (40%). The sera from the mice immunized with the nonmutated constructs (pCI-rev-env wild-type=wt) and positive by ELISA are not neutralizing sera in this assay format.

b—The mouse sera were evaluated for their ability to neutralize the infection of permissive cells with pseudoparticles bearing the wild-type 133 envelope (=nonmutated autolog). The sera from the positive mice and also 2 sera from nonimmunized mice were incubated for 1 h at 37° C. at serial dilutions (from 1/8 to 1/128) with the culture supernatants containing the pseudoparticles. The pseudoparticles bearing the wild-type 133 envelope were obtained in the following way: 293T cells were cotransfected with the construct pCI-env133 and with the plasmid pNL4-3luc (NIH AIDS Research and Reference Reagent Program) with a pNL4-3luc/pCI-env133 ratio of 1/3. This transfection is carried out using the Calcium Phosphate Transfection System Kit (Gibco). The plasmid pNL4-3luc makes it possible to express all the viral genes except vpr and env (frameshift) and also the luciferase gene. Viral pseudoparticles bearing recombinant wild-type 133 envelopes are formed by complementation. Forty eight hours after transfection, the culture supernatants are recovered and centrifuged in order to remove the cell debris. Fetal calf serum is then added to bring the amount to 20%, and the supernatants are aliquotted and stored at −80° C. Assaying of p24, carried out on a VIDAS automated device (bioMérieux), makes it possible to evaluate the amount of viral particles present in these supernatants.

The infection of GHOST-CCR5 or GHOST-CXCR4 cells (NIH AIDS Research and Reference Reagent Program; these cells express CD4) is carried out overnight, in 96-well culture plates, by contact with a volume of supernatant containing the pseudoparticles brought into contact, beforehand, with the sera (10 ng of p24 equivalent per well). Culture medium is then added. After incubation for a further 3 days, the supernatants are removed and the cells are rinsed with 1×PBS. The lysis and the enzyme reaction are carried out with the LucLite Plus kit (Perkin Elmer); the luminescence is read on a TopCount plate reader (Packard Biosciences).

The results show that, for the g12-S3, g14-S3 and g14-S4 sera, slight neutralization is observed for a dilution at 1/8 since, for these samples, the luminescence is approximately 23% less than that recorded for the sera from mice before immunization (V_{g19-S3}=85 000 against V_{T0}=111 000). These results seem to be along the same lines as those described in the preceding experiment. The exception is the g12-S4 serum, for which no neutralizing activity was observed.

### Example 5

### Evaluation of the Functionality of all the Deglycosylation Mutants: Infection with Pseudoviruses and Assays for Neutralization of this Infection with HIV-1-Positive Human Sera.

The obtaining of pseudoparticles expressing the various deglycosylation mutants is important since it makes it possible to study the envelope protein in its natural context, i.e. integrated into a viral particle.

293T cells were cotransfected with each of the mutated pCI-env constructs (deglycosylation mutants, cf. list in table 2) and with the plasmid pNL4-3luc (NIH AIDS Research and Reference Reagent Program) according to the protocol described in example 1 above.

Forty-eight hours after transfection, the culture supernatants are recovered and centrifuged in order to remove the cell debris. Fetal calf serum is then added to bring the amount to 20%, and the supernatants are aliquotted and stored at −80° C. Assaying of p24, carried out on a VIDAS automated device (bioMérieux), makes it possible to evaluate the amount of viral particles present in these supernatants.

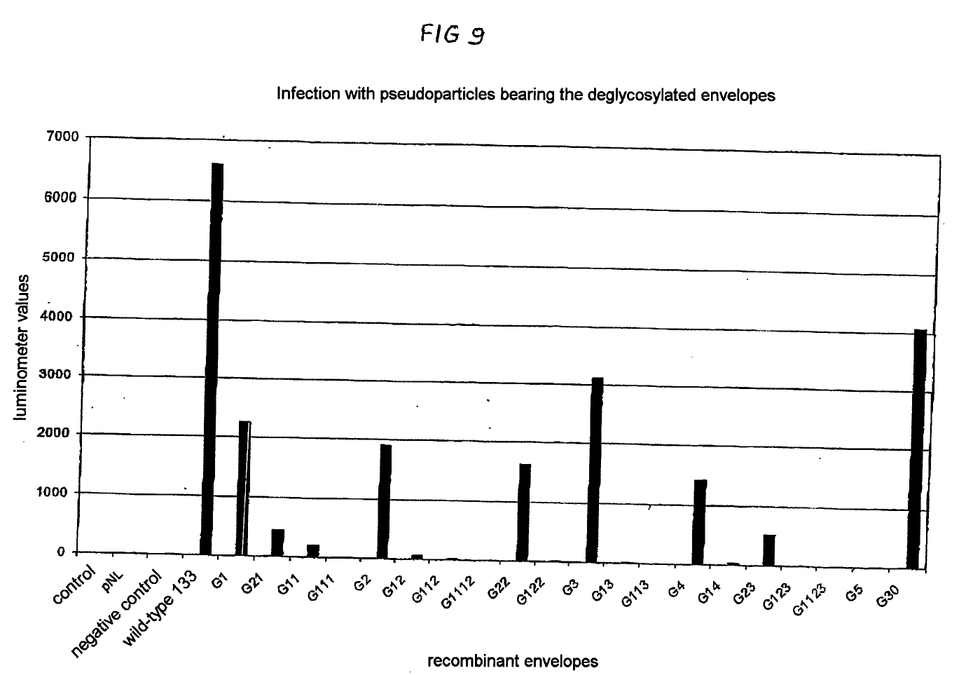

The infection of GHOST-CCR5 or GHOST-CXCR4 cells (NIH AIDS Research and Reference Reagent Program) is carried out overnight, in 24-well culture plates, by contact with a volume of supernatant corresponding to 50 ng of p24 per well. The culture medium is then renewed. After incubation for a further 3 days, the supernatants are removed and the cells are rinsed with 1×PBS and lyzed with 300 μl of CCLR (Cell Culture Lysis Reagent from Promega) for 15 minutes. A volume of 20 μl of these lysates is brought into contact with 80 μl of substrate (Luciferase Assay System from Promega) and the luminescence is measured on a luminometer (Turner Designs TD20/20). The results are given in FIG. 9.

This experiment indicates that, the greater the number of mutations, the more the ability of the recombinant envelopes to induce infection of the GHOST-CCR5 cells decreases. It is zero for the constructs where there is little cleavage or none at all. No mutant induces infection of GHOST-CXCR4 cells. These results are in agreement with the syncytia formation assays previously described (cf. table 4). However, no infection is detected for the g112, g122, g13 and g123 mutants, whereas a slight activity of cell-to-cell fusion was observed. For the g14 mutant used in the neutralization experiments, a small amount of infection is observed. In order to complete this study, we controlled the presence of the envelope protein in the virions. For this, we concentrated and purified the pseudoparticles by ultracentrifugation on a 20% sucrose cushion for 3 h at 70 000 g. The pellets were lyzed in PBS-0.5% Triton and assaying of p24 made it possible to standardize the samples. Western blotting was then carried out using a polyclonal anti-gp120 antibody (Biogenesis). Env is detected only in the pseudoparticles which infect the GHOST cells: when the number of deglycosylated sites is increased, exportation of the envelope protein to the plasma membrane, and also incorporation of the envelope into the pseudoparticles, appear to be affected.

Finally, the sensitivity of these pseudoparticles with respect to neutralizing sera was evaluated: before infection of GHOST-CCR5 indicator cells, the culture supernatants containing the pseudoparticles are incubated for 1 h at 37° C. in the presence of serial dilutions of human serum. This is the GRI/LY serum (a gift from Dr C. Moog, INSERM U544, Strasbourg), the neutralizing titer of which was determined for 5 primary isolates of various subtypes. In parallel, the reference neutralizing HIV-1 sera #1 and #2 (NIBSC) were used as a control on the pseudoparticles bearing the native 133 envelope. The infection protocol is similar to that described above. The infection is carried out in 96-well plates by contact with a volume equivalent to 10 ng of p24. The lysis and the enzyme reaction are carried out with the LucLite Plus kit (Perkin Elmer). The luminescence is read on a TopCount plate reader (Packard Biosciences).

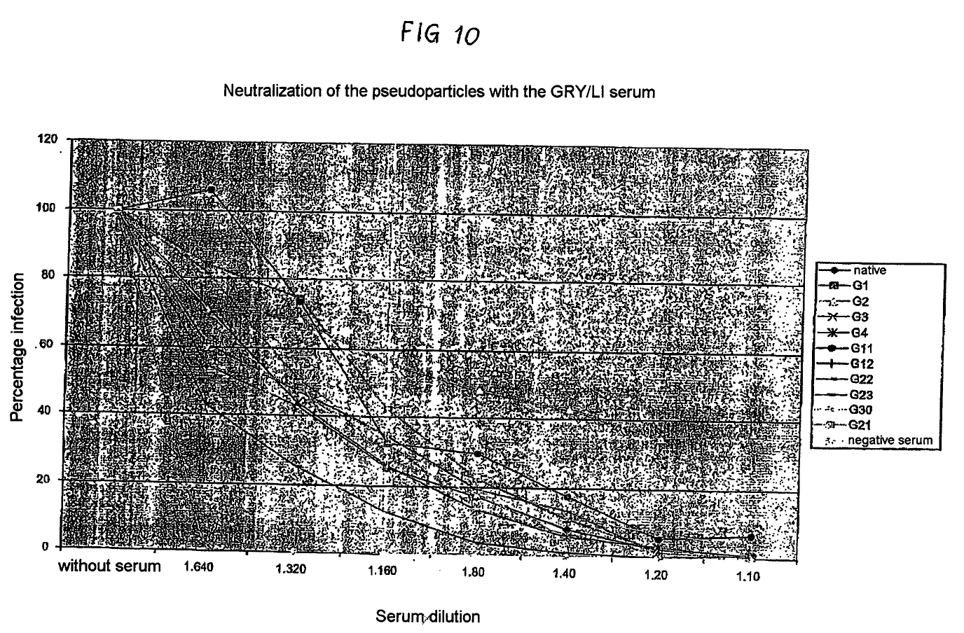

The results are given in FIG. 10.

The “negative serum” curve corresponds to an experiment carried out on pseudovirions expressing the wild-type envelope, brought into contact with an HIV-1 negative serum (CTS Lyon-Gerland). This is the curve which determines the background noise. Neutralization is effective for a serum dilution of greater than 1/640.

These results show that the pseudoparticles are neutralized differently according to the envelope expressed at their surface. However, only with the g30 and g22 mutants is the percentage infection significantly decreased compared to the wild-type (nonmutated) envelope. Even though there is no direct correlation between this experiment and the ability of such a mutant to induce neutralizing antibodies in vivo, the g22 mutant could be an advantageous candidate to be tested for its ability to induce neutralizing antibodies after DNA immunization of small animals.

### BIBLIOGRAPHICAL REFERENCES

1. Moore, J P and Ho, D D, AIDS (1995), 9 suppl. A, pS117-S136
2. Trkola, A et al., Nature (1996) 384 (6605), p184-187
3. Girard, M et al., J. Virol. (1995) 69(10) p6239-6248
4. Poignard, P et al., Immunity (1999) 10(4) p431-438
5. McInerney, TL et al., J. Virol. (1998) 72(2) p1523-1533
6. Reitter, J N, Means RE and Desrosiers R C, Nat. Med (1998) 4(6) p679-684
7. Lee, W R et al., PNAS (1992) 89 p2213-2217
8. Ly A. and Stamatatos L, J. Virol. (1998) 74 p6769-6776
9. Kwong, PD, et al., Nature (1998) 393 (6686) p648-659
10. Zhu, X et al., Biochem (2000) 39 p11194-11204
11. Wyatt, R et al., Nature (1998) 393 (6686) p705-711
12. Kwong, PD et al., Structure (2000) 8 p1329-1339
13. Ataman-Önal, Y et al., AIDS Res. Hum. Retroviruses (1999) 15(11) p1035-1039
14. Trkolla, A et al., J. Virol. (1996) 69 p6609-6617
15. Moullard, M and Decrolly, E, BBA (2000) 1469 p121-132
16. Pollakis, P et al., J. Bio. Chem. (2001) 276 p13433-13441
17. Buchacher, A et al., AIDS Res. & Hum. Ret. (1994) 10 p359-369
18. Moore, JP et al., J. Virol. (1995) 69 p101-109
19. Posner, M et al., J. AIDS (1993) 6 p7-14
20. Thali, M et al., J. Virol. (1992) 66 p5635-5641
21. Gershoni, J M et al., FASEB J. (1993) 7 p1185-1187
22. Wain-Hobson, L et al., Cell (1985) 40 p9-17
23. Ratner, L et al., Nature (1985) 313 p277-284
24. Muesing, M A et al., Nature (1985) 313 p450-458
25. Sanchez-Pescador et al., Science (1985) 227 p484-492.

1. A mutated env gene encoding a mutated envelope glycoprotein of the HIV-1 virus, characterized in that said gene exhibits, compared to the env gene of a “reference” primary infection primary isolate, at least two mutations at the glycosylation sites conserved from one primary isolate to another, each mutation consisting of replacement of an AAC or AAT codon which encodes an asparagine with a CAG or CAA codon which encodes a glutamine, the two mutations at least being chosen from the following:
(a) at least two mutations in the part of the env gene encoding the C3 region of the env protein,
(b) at least one mutation in the part encoding the C3 region and at least one mutation in the part encoding the V3 region,
(c) at least one mutation in the part encoding the C2 region and at least one mutation in the part encoding the V3 region chosen from the glycosylation sites at codons 862-864 and 970-972,
(d) at least one mutation in the part encoding the C2 region chosen from the glycosylation sites at codons 667-669, 679-681, 700-702, 763-765 and 844-846, and at least one mutation in the part encoding the V3 region,
it being possible for the position of at least any one of said codons to vary by three to twenty-four nucleotides,
or the mutated env gene consists of any one of the sequences SEQ ID NO: 8, SEQ ID NO: 9 or SEQ ID NO: 10 or the sequences complementary thereto.

2. The gene as claimed in claim 1, characterized in that, according to the mutations (a), at least one mutation is effected at codons 976-978, 991-993 of the part encoding the C3 region and at least one mutation is effected at codons 1039-1041 or 1060-1062 of the part encoding the C3 region, it being possible for the position of at least any one of said codons to vary by three to twenty-four nucleotides.

3. The gene as claimed in claim 2, characterized in that its sequence consists of a sequence chosen from the sequences identified in SEQ ID NO: 21, SEQ ID NO: 22 and SEQ ID NO:23, or the sequences complementary thereto.

4. The gene as claimed in claim 1, characterized in that, according to the mutations (b), at least one mutation is effected at codons 976-978, 991-993, 1.039-1041 or 1060-1062 of the part encoding the C3 region and at least one mutation is effected at codon 880-882 of the part encoding the V3 region, it being possible for the position of at least any one of said codons to vary by three to twenty-four nucleotides.

5. The gene as claimed in claim 4, characterized in that the mutations are effected at codons 976-978, 991-993 and 880-882, it being possible for the position of at least any one of said codons to vary by three to twenty-four nucleotides.

6. The gene as claimed in claim 5, characterized in that its sequence consists of a sequence chosen from the sequences identified in SEQ ID NO: 2, SEQ ID NO: 3 and SEQ ID NO: 4, or the sequences complementary thereto.

7. The gene as claimed in claim 4, characterized in that at least one mutation is effected at codon 976-978 or 991-993, at least one mutation is effected at codon 880-882, and at least one mutation is effected at codon 1039-1041 or 1060-1062, it being possible for the position of at least any one of said codons to vary by three to twenty-four nucleotides.

8. The gene as claimed in claim 7, characterized in that the mutations are effected at codons 976-978, 991-993, 880-882, 1039-1041 and 1060-1062.

9. The gene as claimed in claim 8, characterized in that its sequence consists of a sequence chosen from the sequences identified in SEQ ID NO: 5, SEQ ID NO: 6 and SEQ ID NO: 7, or the sequences complementary thereto.

10. The gene as claimed in claim 1, characterized in that, according to the mutations (c), at least one mutation is effected at codon 805-807 of the part encoding the C2 region, it being possible for at least any one of said codons to vary by three to twenty-four nucleotides.

11. The gene as claimed in claim 1, characterized in that according to the mutations (d), at least one mutation is effected at codon 880-882 of the part encoding the V3 region, it being possible for the position of at least any one of said codons to vary by three to twenty-four nucleotides.

12. A mutated Env glycoprotein of the HIV-1 virus, characterized in that it exhibits, compared to a native Env protein of a “reference” primary infection primary isolate, at least two mutations at the glycosylation sites of said reference protein which are conserved from one primary isolate to another, each mutation consisting of replacement of an asparagine with a glutamine, the two mutations at least being chosen from the following:
(a′) at least two mutations in the C3 region of the Env protein,
(b′) at least one mutation in the C3 region and at least one mutation in the V3 region,
(c′) at least one mutation in the C2 region and at least one mutation in the V3 region chosen from the glycosylation sites at amino acid 288 or 324,
(d′) at least one mutation in the C2 region chosen from the glycosylation sites at any one of amino acids 223, 227, 234, 255 and 282, and at least one mutation in the V3 region,
it being possible for the position of said glycosylation sites conserved from one primary infection primary isolate to another to vary by one to eight amino acids,
or the mutated Env glycoprotein consists of any one of the sequences or the mutated env gene consists of any one of the sequences SEQ ID NO: 18, SEQ ID NO: 19 or SEQ ID NO: 20.

13. The glycoprotein as claimed in claim 12, characterized in that, according to the mutations (a′), at least one mutation is effected at the glycosylation site at amino acid 326 or 331 and at least one mutation is effected at the glycosylation site at amino acid 347 or 354,
it being possible for the position of said glycosylation sites conserved from one primary infection primary isolate to another to vary by one to eight amino acids.

14. The glycoprotein as claimed in claim 13, characterized in that its sequence consists of a sequence chosen from the sequences identified in SEQ ID NO: 21, SEQ ID NO: 22 and SEQ ID NO: 23.

15. The glycoprotein as claimed in claim 12, characterized in that, according to the mutations (b′), at least one mutation is effected at amino acid 326 or 331 and at least one mutation is effected at amino acid 294,
it being possible for the position of at least any one of said glycosylation sites conserved from one primary infection primary isolate to another to vary by one to eight amino acids.

16. The glycoprotein as claimed in claim 15, characterized in that the mutations are effected at amino acids 326, 331 and 294,
it being possible for the position of at least any one of said glycosylation sites conserved from one primary infection primary isolate to another to vary by one to eight amino acids.

17. The glycoprotein as claimed in claim 16, characterized in that its sequence consists of a sequence chosen from the sequences identified in SEQ ID NO: 12, SEQ ID NO: 13 and SEQ ID NO: 14.

18. The glycoprotein as claimed in claim 12, characterized in that, according to the mutations (b′), at least one mutation is effected at amino acid 326 or 331, at least one mutation is effected at amino acid 347 or 354, and at least one mutation is effected at amino acid 294,
it being possible for the position of at least any one of said glycosylation sites conserved from one primary infection primary isolate to another to vary by one to eight amino acids.

19. The glycoprotein as claimed in claim 18, characterized in that the mutations are effected at amino acids 326, 331, 294, 347 and 354,
it being possible for the position of at least any one of said glycosylation sites conserved from one primary infection primary isolate to another to vary by one to eight amino acids.

20. The glycoprotein as claimed in claim 19, characterized in that its sequence consists of a sequence chosen from the sequences identified in SEQ ID NO: 15, SEQ ID NO: 16 and SEQ ID NO: 17.

21. The glycoprotein as claimed in claim 12, characterized in that, according to the mutations (c′), at least one mutation is effected at amino acid 269 of the C2 region, it being possible for the position of said glycosylation sites conserved from one primary infection primary isolate to another to vary by one to eight amino acids.

22. The glycoprotein as claimed in claim 12, characterized in that, according to the mutations (d′), at least one mutation is effected at amino acid 294 of the V3 region, it being possible for the position of said glycosylation sites conserved from one primary infection primary isolate to another to vary by one to eight amino acids.

23. A pharmaceutical composition comprising:
(i) at least one mutated gene encoding a mutated envelope glycoprotein of the HIV-1 virus, said mutated gene being chosen from any one of the genes described in any one of claims 1 to 11 and being placed under the control of regulatory sequences which allow its expression in a host cell,
a pharmaceutically acceptable vehicle, and
optionally, an additional agent which facilitates the penetration of said mutated gene into said cell and/or which makes it possible to target said cell, or
(ii) at least the gene identified in (i) cloned into a recombinant viral vector.

24. A pharmaceutical composition comprising:
at least one mutated envelope glycoprotein of the HIV-1 virus, said mutated glycoprotein being chosen from any one of the glycoproteins described in any one of claims 12 to 22,
a pharmaceutically acceptable vehicle and/or excipient and/or adjuvant and/or diluent.

25. A method for obtaining antibodies, according to which a mammalian animal, such as a mouse, rat or monkey, is immunized with at least one mutated gene encoding a mutated envelope glycoprotein of the HIV-1 virus, said mutated gene being chosen from any one of the genes described in any one of claims 1 to 11 and being placed under the control of regulatory sequences which allow its expression in vivo.

26. A method for obtaining antibodies, according to which a mammalian animal, such as a mouse, rat or monkey, is immunized with at least one mutated envelope glycoprotein of the HIV-1 virus, said mutated glycoprotein being chosen from any one of the glycoproteins described in any one of claims 12 to 22.

27. An antibody obtained according to the method of claim 25 or 26.

28. A pharmaceutical composition comprising at least one antibody as claimed in claim 27.

29. A method for evaluating a therapeutic agent, according to which at least one mutated gene encoding a mutated envelope glycoprotein of the HIV-1 virus as described in any one of claims 1 to 11 is administered to an animal, and the following are carried out:
measurement of the titer of antibodies specific for the mutated glycoprotein, and/or
measurement of the titer of neutralizing antibodies specific for the mutated or native glycoprotein, and/or
measurement of the cellular immune response induced against the mutated glycoprotein, for example using an assay for the in vitro activation of T “helper” lymphocyte cells specific for the mutated glycoprotein.
